(19) United States
(12) Patent Application Publication
(10) Pub. No.: US 2025/0144356 A1
(43) Pub. Date: May 8, 2025

(54) VALVED HME ASSEMBLIES FOR USE IN RESPIRATORY CIRCUITS

(71) Applicant: AEON RESEARCH AND TECHNOLOGY, INC., Hewlett, NY (US)

(72) Inventors: Sunil Kumar Dhuper, Old Westbury, NY (US); Greg Marler, Rockford, IL (US)

(73) Assignee: AEON RESEARCH AND TECHNOLOGY, INC., Hewlett, NY (US)

(21) Appl. No.: 18/934,866

(22) Filed: Nov. 1, 2024

Related U.S. Application Data
(60) Provisional application No. 63/596,025, filed on Nov. 3, 2023.

(51) Int. Cl. A61M 16/10; A61M 16/20

(52) U.S. Cl. CPC A61M 16/1045; A61M 16/208; A61M 2202/0007; A61M 2202/02

(57) ABSTRACT

An HME unit for use in a breathing circuit is provided and includes an HME chamber that contains an HME member. The HME unit also includes an inner core for reception in the HME chamber. The inner core having at least one first opening formed therein and at least one second opening formed therein and spaced from the at least one first opening with an intermediate area being located between the at least one first opening and the at least one second opening. The HME member is disposed about the intermediate area of the inner core. The HME unit also includes a movable valve that has a valve member disposed within an interior bore of the inner core and being movable between a fully open position and a fully closed position that causes incoming breathing gas to flow across the HME member.

### CROSS-REFERENCE TO RELATED PATENT APPLICATIONS

This application is based on and claims priority to U.S. Provisional Patent Application 63/596,025, filed Nov. 3, 2023, the entire contents of which is incorporated by reference herein as if expressly set forth in its respective entirety herein.

### TECHNICAL FIELD

The present invention relates to respiratory circuits and more particularly, to valved heat moisture exchangers (HMEs) that have a plurality of different operating modes including an HME mode, an HME bypass mode and a mode in which gas flows along two paths one which passes through the HME and the other which bypasses the HME.

### BACKGROUND

There are many types of breathing circuits that are designed to deliver breathing gas (e.g., oxygen) to patients. One consideration in such breathing circuit design is the inclusion of a heat and moisture exchanger (HME). As is known, a heat and moisture exchanger is a device that is used in mechanically ventilated patients intended to help prevent complications due to drying of the respiratory mucosa, such as mucus plugging and endotracheal tube (ETT) occlusion. The HME is typically formed of a layer of foam or paper embedded with a hydroscopic salt, such as calcium chloride.

Traditional HME bypass systems are designed so that the HME pathway is either closed or open depending upon the position of a valve that is disposed within the HME pathway. In any event, the HME bypass is designed based on the principle of opening and closing the HME pathway itself.

### SUMMARY

In one embodiment, an HME unit for use in a breathing circuit is provided. The HME unit includes a first conduit and an HME chamber that contains an HME member. The first conduit is in fluid communication with the HME chamber. A second conduit is in fluid communication with the HME chamber. The HME unit also includes an inner core for reception in the HME chamber. The inner core having at least one first opening formed therein and at least one second opening formed therein and spaced from the at least one first opening with an intermediate area being located between the at least one first opening and the at least one second opening. The HME member is disposed about the intermediate area of the inner core. The HME unit also includes a movable valve that has a valve member disposed within an interior bore of the inner core and being movable between a fully open position and a fully closed position in which the valve member occludes the interior bore of the inner core and is configured to force incoming breathing gas to flow through the at least one first opening across the HME member before flowing back through the at least one second opening and subsequently into the second conduit.

In contrast to traditional HME bypass systems, the present systems are designed so that the HME pathway is always open since there is no valve or the like within the HME pathway. Instead, the valve is located within the main fluid pathway and not the HME pathway. The HME pathway is never blocked and remains always open regardless of the mode of operation. Accordingly, the present systems can be thought of as being a main pathway bypass system. Thus, the flow into the HME is controlled by the main pathway flow characteristics. In addition, certain embodiments are described herein in which both the main delivery pathway and the HME pathway are open; however, even when both pathways are open, flow dynamics will cause substantially all or nearly all of the medication to flow along the main flowpath even if the HME pathway is open. As a result, in at least some embodiments, even if the user does not set the valve in the proper position and the HME flowpath remains open, virtually all of the medication is delivered to the patient along the main flowpath.

### BRIEF DESCRIPTION OF THE DRAWING FIGURES

FIG. 1 is side elevation of an HME unit according to a first embodiment;

FIG. 2 is a cross-sectional view of the HME unit of FIG. 1;

FIG. 3 is a cross-sectional view of the HME in which a main flow path is closed and an HME flow path is open;

FIG. 4A is a cross-sectional view of the HME in which the main flow path is open and the HME flow path is closed;

FIG. 4B is a cross-sectional view of the HME in which the main flow path is open and the HME flow path is open;

FIG. 5 is an enlarged sectional view showing a valve stop;

FIG. 6 is an exploded perspective view of the HME unit;

FIG. 7 is side elevation of an HME unit according to a second embodiment;

FIG. 8 is a cross-sectional view of the HME unit of FIG. 7;

FIG. 9 is a cross-sectional view of the HME in which a main flow path is closed and an HME flow path is open;

FIG. 10 is a cross-sectional view of the HME in which the main flow path is open and the HME flow path is open;

FIG. 11 is an enlarged sectional view showing a valve stop;

FIG. 12 is an exploded perspective view of the HME unit;

FIG. 13 is side elevation of an HME unit according to a third embodiment;

FIG. 14 is a cross-sectional view of the HME in which a main flow path is closed and an HME flow path is open;

FIG. 15 is a cross-sectional view of the HME in which the main flow path is open and the HME flow path is closed;

FIG. 16 is a cross-sectional perspective view in which the main flow path is closed;

FIG. 17 is a cross-sectional perspective view in which the main flow path is open;

FIG. 18 is a perspective view of the HME unit showing a nozzle thereof;

FIG. 19 is an exploded perspective view thereof;

FIG. 20 is side elevation of an HME unit according to a fourth embodiment;

FIG. 21 is a cross-sectional view of the HME in which an HME flow path is open;

FIG. 22 is a cross-sectional view of the HME in which a main flow path is open and the HME flow path is open;

FIG. 23 is an end view showing the valve in an open position whereby the main flow path is open;

FIG. 24 is an end view showing the valve in a closed position whereby the main flow path is closed;

FIG. 25 is an exploded view of the HME unit;

FIG. 26 is cross-sectional view of an HME unit according to a fifth embodiment in which a main flow path is closed and the HME flow path is open;

FIG. 27 is a cross-sectional view of the HME unit in which a main flow path is open and the HME flow path is closed;

FIG. 28 is a perspective view the HME unit;

FIG. 29 is another perspective view of the HME unit;

FIG. 30 is cross-sectional view of an HME unit according to a sixth embodiment in which a main flow path is closed and the HME flow path is open;

FIG. 31 is a cross-sectional view of the HME unit in which a main flow path is open and the HME flow path is closed;

FIG. 32 is another cross-sectional view the HME unit;

FIG. 33 is a top plan view of the HME unit;

FIG. 34 is cross-sectional view of an HME unit according to a seventh embodiment in which a main flow path is closed and the HME flow path is open;

FIG. 35 is a top plan view of the HME unit;

FIG. 36 is an end cross-sectional perspective view in which the valve member is closed and the main flow path is closed;

FIG. 37 is an end cross-sectional perspective view in which the valve member is open and the main flow path is closed;

FIG. 38 is a side perspective view of an HME unit according to an eighth embodiment with expandable/collapsible corrugated tubing in a collapsed state;

FIG. 39 is a side perspective view of the HME unit of FIG. 38 with the expandable/collapsible corrugated tubing in an expanded state;

FIG. 40 is an exploded perspective view an HME unit that is similar to that shown in FIG. 38;

FIG. 41 is an exploded perspective, in cross-section, of the HME unit;

FIG. 42 is a cross-sectional perspective view of a first part of the HME unit showing a valve member in a closed position;

FIG. 43 is a cross-sectional perspective view of the first part of the HME unit showing the valve member in an open position;

FIG. 44 is a cross-sectional perspective view of the first part of the HME unit showing the valve member in an open position;

FIG. 45 is a cross-sectional perspective view of the first part of the HME unit showing the valve member in the open position;

FIG. 46 is an end view of one part of the HME unit showing HME openings that communicate with the HME media;

FIG. 47 is a side perspective view of internal components of the one part shown in FIG. 46; and

FIG. 48 is a perspective view of an end cap of the first part of FIG. 46.

### DETAILED DESCRIPTION OF CERTAIN EMBODIMENTS

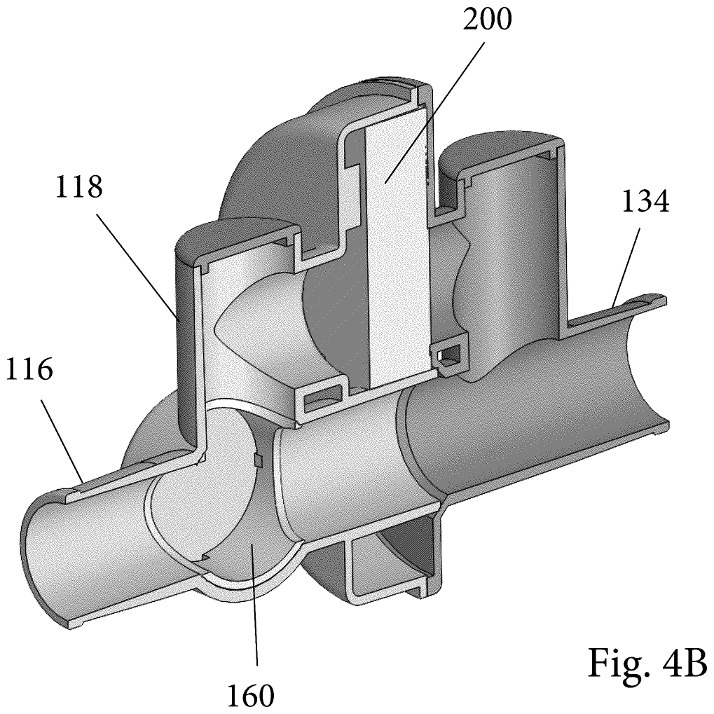

As described herein, the present disclosure is directed to valved HME units that are intended for placement in respiratory circuits and are configured to provide a plurality of different flow paths including one which direct fluid (gas) through an HME and another in which the fluid bypasses the HME and flows to the patient. Some of the embodiments also include an operating mode in which the fluid can flow both through the HME and along the bypass flow path (See, FIG. 4B).

As is known, a heat and moisture exchanger (HME) is a device that is used in mechanically ventilated patients intended to help prevent complications due to drying of the respiratory mucosa, such as mucus plugging and endotracheal tube (ETT) occlusion. The HME is typically formed of a layer of foam or paper embedded with a hydroscopic salt, such as calcium chloride. In respiratory circuits, it is thus desirable for breathing gas (air and/or supplemental oxygen) to pass through an HME before being delivered to the patent and also the exhaled air passes through the HME to retain moisture in the exhaled gas. However, when a medication or other agent is required to be delivered to the patient, it is desirable to bypass the HME since if the medication passes into the HME, the medication will be attracted to and saturate the HME. It is therefore desirable to configure the HME so that the fluid can flow to the patient along multiple different selectable flow paths one of which is a flow path that bypasses the HME.

### HME Unit According to First Embodiment

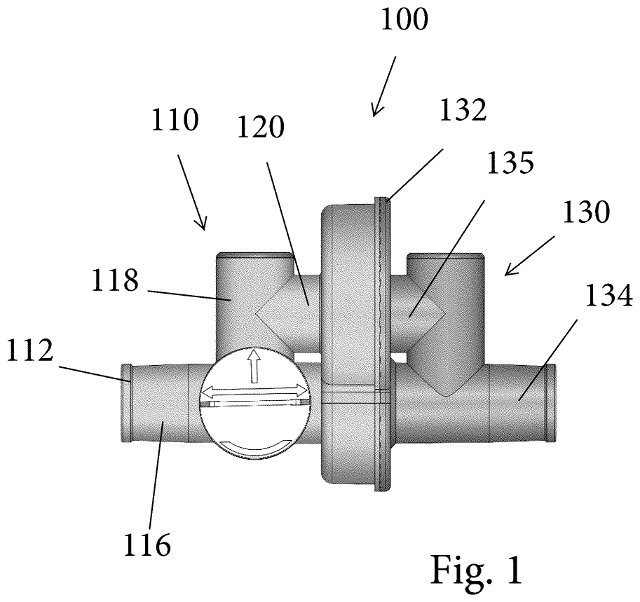

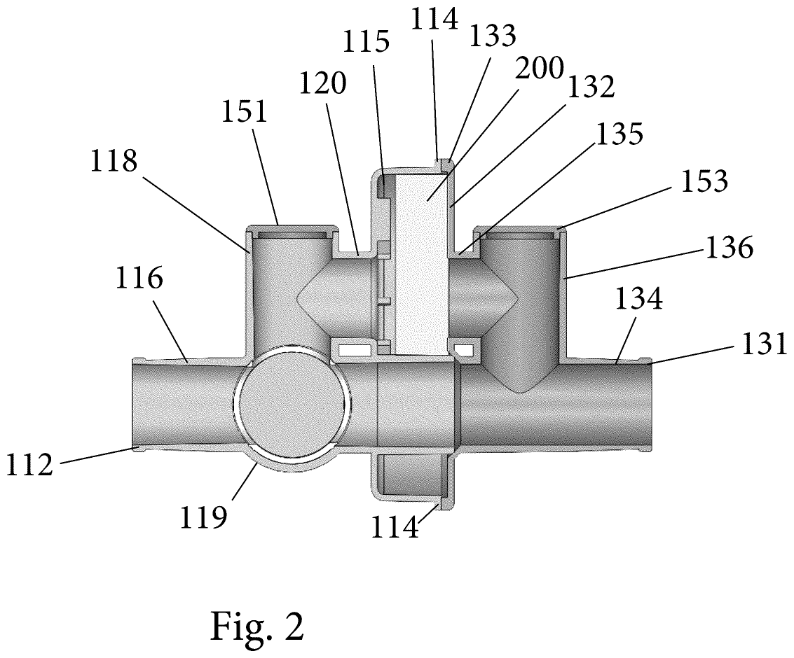

FIGS. 1-6 illustrate an HME unit 100 according to a first embodiment. The HME unit 100 includes a housing that is defined by a first housing part 110 and a second housing part 130 that are coupled to one another to define a complete assembled housing. As shown and as described herein, the housing is a hollow structure that has a number of conduits that define flow paths and at least one compartment (space).

The first housing part 110 has a first end 112 and an opposite second end 114 with the first end 112 being an end that receives a fluid (e.g., gas) that is to be delivered to the patient and the second end 114 being the end that is coupled to the second housing part 130. At the first end 112, the first housing part 110 has a first conduit section 116 which is a main conduit section of the first housing part 110. A second conduit section 118 branches off of the first conduit section 116 at a first location. As shown the second conduit section 118 can be formed at a right angle relative to the first conduit section 116 and is in direct fluid communication with the first conduit section 116. At the first location, the first housing part 110 includes a valve conduit section 119 that is in communication with the first conduit section 116. The valve conduit section 119 can be formed at a right angle relative to the first conduit section 116. In addition, a first connector conduit 120 is provided and extends outwardly from the second conduit section 118 and runs parallel to the first conduit section 116. One end of the first connector conduit 120 is thus in fluid communication with the interior of the second conduit section 118.

As shown in the figures, a portion of the first conduit section 116 is located downstream of the valve conduit section 119.

The first housing part 110 also includes an end compartment 115 at the second end 114. The end compartment 115 is in fluid communication with one end of the first connector conduit 120 and is located above one end of the first conduit section 116. The end compartment 115 of the housing defines a hollow interior space that receives and holds an HME 200 as described below. The end compartment 115 is thus designed so that the first connector conduit 120 opens into the end compartment 115 and thus allows gas to be directed to the HME 200. As shown in the figures, the end compartment 115 is located above the end portion of the first conduit section 116.

At a second end 133, the second housing part 130 has an end plate 132 that is configured to mate with the second end 114 of the first housing part 110. At a first end 131, the second housing part 130 has a first conduit section 134 which is a main conduit section of the second housing part 130. A second conduit section 136 branches off of the first conduit section 134 at a first location before the end plate 132. As shown, the second conduit section 136 can be formed at a right angle relative to the first conduit section 134 and is in direct fluid communication with the first conduit section 134. The first conduit section 134 extends to the end plate 132 at which an opening is formed to allow direct access to the first conduit section 134. In addition, a second connector conduit 135 is provided and extends outwardly from the second conduit section 136 and runs parallel to the first conduit section 134. One end of the second connector conduit 135 is thus in fluid communication with the interior of the second conduit section 136. An opposite end of the second connector conduit 135 extends to the end plate 132 at which an opening is formed to allow direct access to the second connector conduit 135. With the end plate 132, the entrance to the second connector conduit 135 is thus directly above the entrance to the first conduit section 134.

It will be appreciated that shown, the sizes (diameters) of the various conduit sections described above can be different. The first connector conduit and the second connector conduit in one embodiment have the same dimensions (e.g., same diameter).

The HME 200 is designed to be held and contained within the end compartment 115. The HME 200 is designed so cover the open end of the first connector conduit 120 so that fluid flowing within the first connector conduit 120 is forced into contact with the HME 200. The size and shape of the HME 200 are thus selected in view of the size and shaped of the end compartment 115. As can be seen in the figures, in the illustrated embodiment, the HME 200 has a generally circular shaped body with a bottom cutout or notch in the form of a partial circle. In other words, the bottom edge of the HME 200 has an arcuate shaped notch that is sized and shaped to receive a curved top of the first conduit section 134. The HME 200 can thus nest and seat over the curved first conduit section 134.

Any number of traditional techniques can be used to attach the first housing part 110 to the second housing part 130. Since the HME 200 needs periodic replacement, the first and second housing parts 110, 130 are attached in a way that allows for separation thereof.

A first cap 151 can be provided for capping off the top end of the second conduit section 118 of the first part and a second cap 153 can be provided for capping of the top end of the second conduit section 136 of the second part. The caps 151, 153 are preferably removable.

A valve member 160 is provided and can be manipulated by a user to select one of a number of valve operating positions. The valve member 160 is configured to be disposed within the valve conduit section 119 and be rotatable therein. The valve member 160 has a cylindrical shaped body and includes an outer wall 162 on which indicia can be provided to indicate the different positions of the valve member 160. The cylindrical shaped body includes three openings that are formed circumferentially around the cylindrical shaped body. For example, there is a pair of first openings that are formed 180 degrees apart and a second opening that is spaced 90 degrees from each of the first openings. These three openings thus extend along 270 degrees of the cylindrical shaped body.

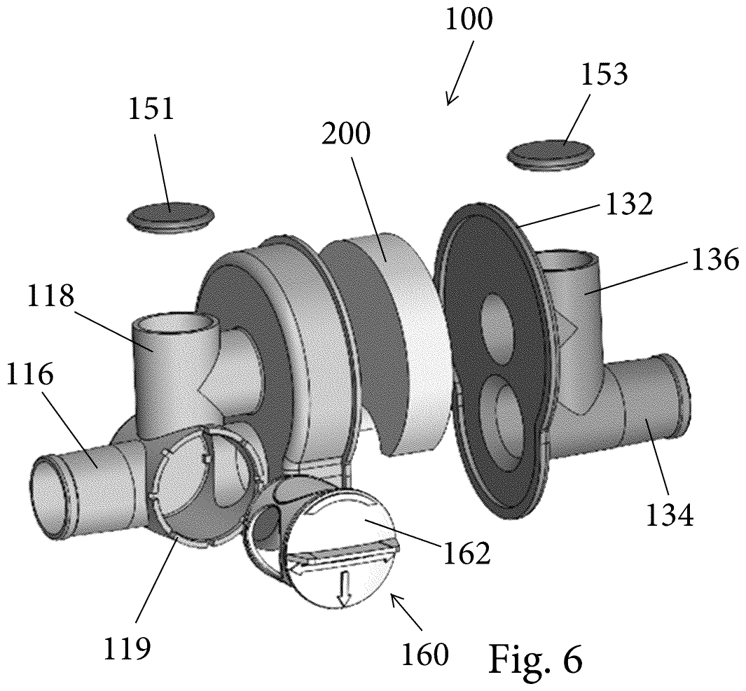

The valve member 160 has three distinct positions or settings. A first valve setting is on in which the pair of first openings are aligned with the first conduit section 116 and the second opening faces downward away from the second conduit section 118. In this setting, only the first conduit section 116 is open and the second conduit section 118 is closed off from the first conduit section and thus, fluid cannot flow into the second conduit section 118. Instead, fluid entering the first conduit section 116 flows longitudinally (linear) within the first conduit section 116 to the first conduit section 134 of the second housing part 130. This position is shown in FIGS. 4A and 6. This position can be thought of as being an HME bypass valve position.

In the embodiment of FIG. 4B both the main flow path and the HME pathway are open due to the position of the valve member 160. As shown, the valve member 160 is rotated with the pair of openings of the valve member 160 axially aligned with the first conduit section 116 and the first conduit section 134. At the same time, the other opening formed in the valve member 160 that is 90 degrees offset from the pair of openings is aligned with the second conduit section that defines the HME path. As a result, incoming fluid can flow both axially along the main flow path and also can flow along the HME path.

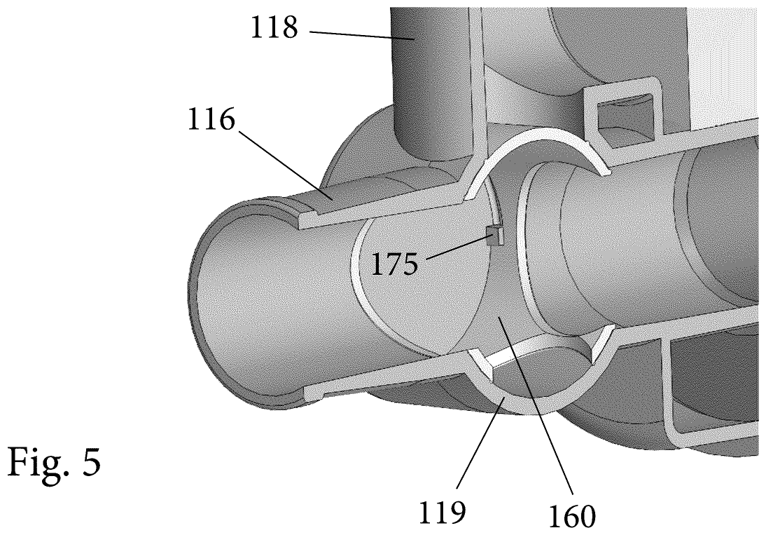

As shown in FIG. 5, the rotation of the valve member 160 can be limited by a mechanism that allows for rotation of the valve only a certain number of degrees. For example, an inner edge of the valve member 160 that is inserted into the valve conduit 119 (valve cradle section) can have a circumferential notch formed therein. This notch extends a prescribed number of degrees. There is a complementary standoff (protrusion/tab) 175 that is formed in the valve cradle. The standoff 175 is received within the notch and the valve member 160 can only rotate a prescribed number of degrees. The degree of movement of the valve member 160 is between one position in which the standoff 175 contacts one end of the notch and another position in which the standoff 175 contact the other end of the notch. The arcuate length of the notch defines the degree of travel.

As best shown in FIG. 6, the end plate 132 has a pair of openings formed therein, one for the second connector conduit 135 and the other for the first conduit section 134.

A first flow path is thus defined when the valve member 160 is in the first valve position. In the first flow path, the fluid enters the first conduit section 116 of the first housing part and flows linearly into the first conduit section 134 of the second housing part 130 and then can flow to the patient via a patient interface (e.g., mask or breathing tube) that is fluidly coupled to the second housing part 130.

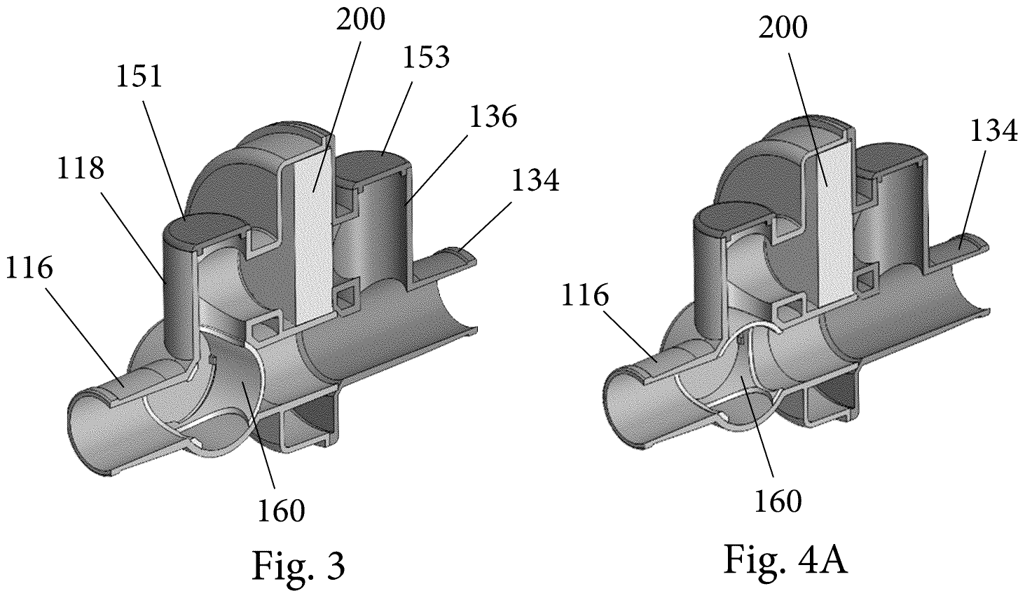

In a second valve setting, shown in FIG. 3, the valve member 160 is positioned such that one of the first openings is aligned with the second conduit section 118 and the second opening is aligned with the first conduit section 116 to allow the fluid entering the first conduit section 116 to flow exclusively into the second conduit section 118. The portion of the valve member 160 that is opposite the second opening is solid and thus fluid is not permitted to flow along the first conduit section 116 to the first conduit section 134 of the second housing part 130. This position can be thought of as being an HME position all of the fluid entering into the first conduit section 134 is directed into the second conduit section 118 and passes through the HME 200.

A second flow path is thus defined when the valve member 160 is in the second valve position. In the second flow path, the fluid enters the first conduit section 116 of the first housing part and flows at a right angle into the second conduit section 118 and then flows into the first connector conduit 120. As the fluid flows along the first connector conduit 120, the fluid flows across the HME 200 and into the second connector conduit 135 before then flowing into the second conduit section 136 and then finally into the first conduit section 134 of the second housing part 130. In this setting, all of the fluid entering the first housing part is directed through the HME to provide HME treated fluid (breathing gas). Exhaled air can follow the reverse flow and go from the second housing part through the HME 200 to the first housing part.

There is also an optional third valve setting in which the valve member 160 is positioned such that the pair of openings are open and aligned with the first conduit section 116 and at the same time the second opening in the valve member 160 is aligned with and open to the second conduit section 118. In this third setting, some fluid can bypass the HME 200 while some fluid can be directed across the HME 200.

During normal breathing and when no medication is to be delivered, the valve member 160 can be positioned in the second valve position and the fluid is directly exclusively along the second flow path and is conducted across the HME 200 and then subsequently is delivered to the patient. In the event that medication needs to be delivered to the patient, the valve member 160 is placed in the first valve position (HME bypass) and the medication enriched fluid bypasses the HME 200 and flows to the patient.

### HME Unit According to Second Embodiment

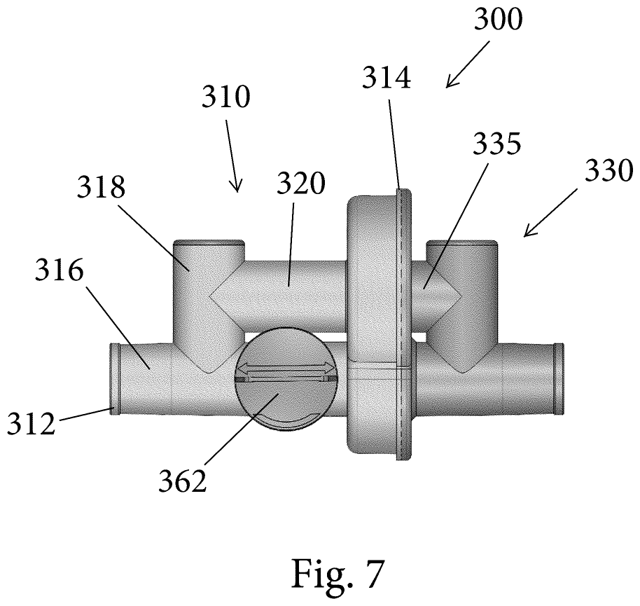

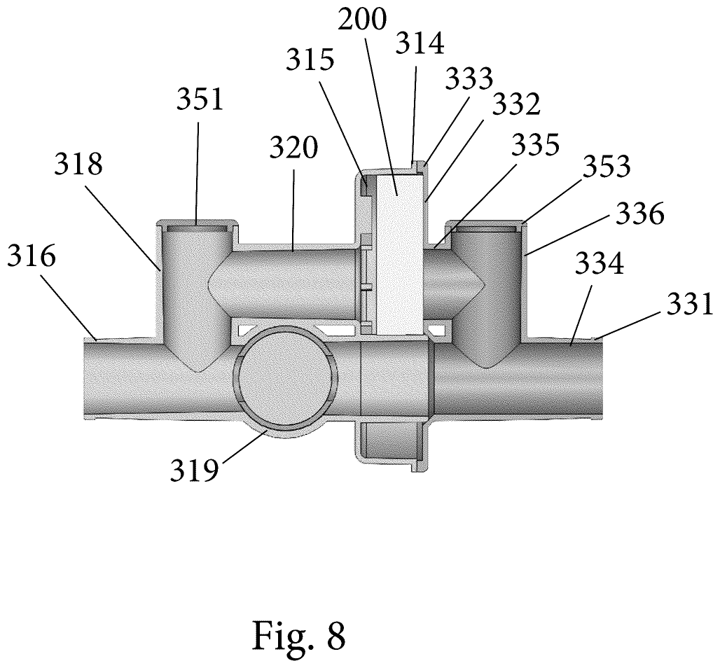

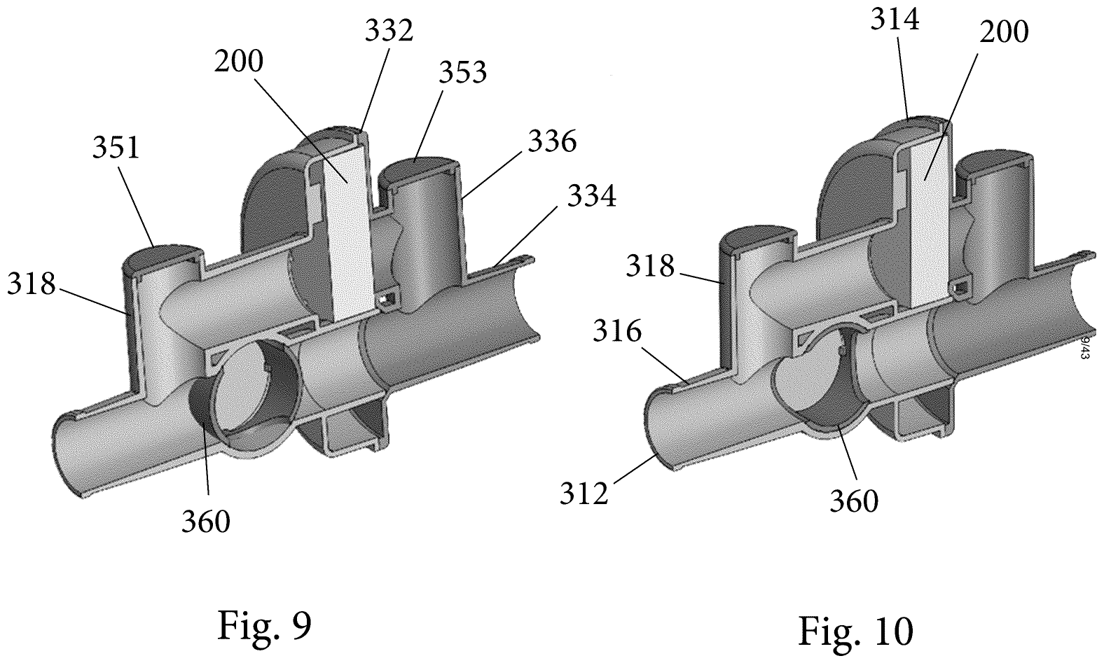

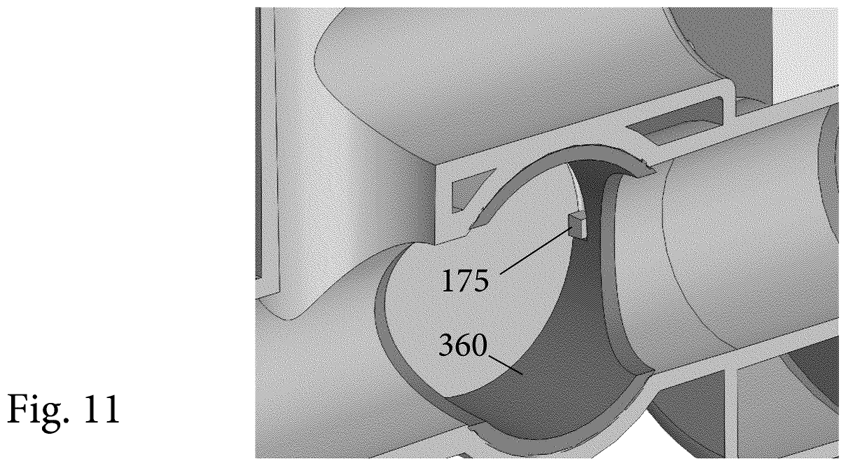

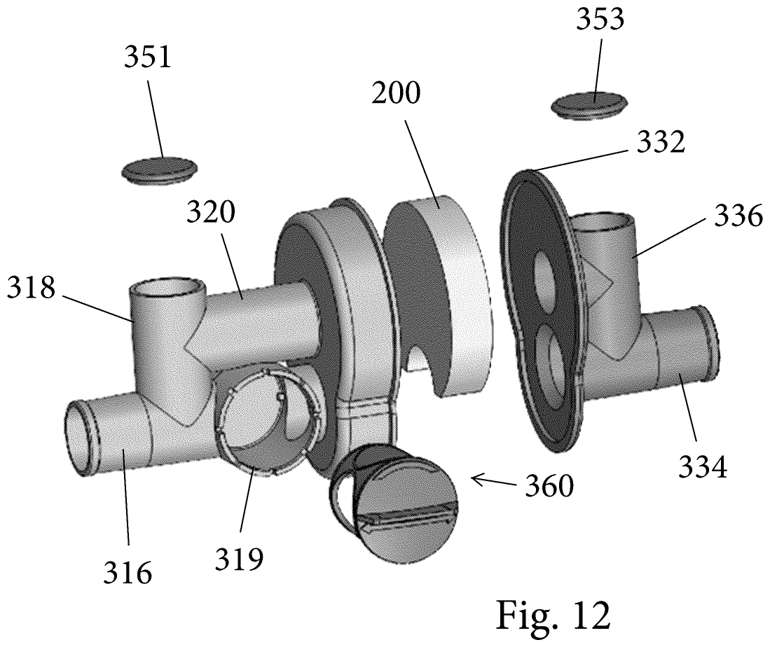

FIGS. 7-12 illustrate an HME unit 300 according to a second embodiment that is similar to the HME unit 100. The HME unit 300 includes a housing that is defined by a first housing part 310 and a second housing part 330 that are coupled to one another to define a complete assembled housing. As shown and as described herein, the housing is a hollow structure that has a number of conduits that define flow paths and at least one compartment (space).

The first housing part 310 has a first end 312 and an opposite second end 314 with the first end 312 being an end that receives a fluid (e.g., gas) that is to be delivered to the patient and the second end 314 being the end that is coupled to the second housing part 330. At the first end 312, the first housing part 310 has a first conduit section 316 which is a main conduit section of the first housing part 310. A second conduit section 318 branches off of the first conduit section 316 at a first location. As shown the second conduit section 318 can be formed at a right angle relative to the first conduit section 316 and is in direct fluid communication with the first conduit section 316. At the first location, the first housing part 310 includes a valve conduit section 319 that is in communication with the first conduit section 316. The valve conduit section 319 can be formed at a right angle relative to the first conduit section 316.

Unlike the first embodiment in which the second conduit section 118 is located immediately above the valve conduit section 119, the second conduit section 318 is formed upstream of the valve conduit section 319 and is not directly above the valve conduit section 319.

In addition, a first connector conduit 320 is provided and extends outwardly from the second conduit section 318 and runs parallel to the first conduit section 316. One end of the first connector conduit 320 is thus in fluid communication with the interior of the second conduit section 318.

As shown in the figures, a portion of the first conduit section 316 is located downstream of the valve conduit section 319.

The first housing part 310 also includes an end compartment 315 at the second end 314. The end compartment 315 is in fluid communication with one end of the first connector conduit 320 and is located above one end of the first conduit section 316. The end compartment 315 of the housing defines a hollow interior space that receives and holds the HME 200. The end compartment 315 is thus designed so that the first connector conduit 320 opens into the end compartment 315 and thus allows gas to be directed to the HME 200. As shown in the figures, the end compartment 315 is located above the end portion of the first conduit section 316.

The first housing part 310 is thus configured such that the second conduit section is located upstream of the valve mechanism that is disposed in the valve conduit section 319 and therefore, fluid can flow into the second conduit section prior to contacting the valve mechanism.

At a second end 333, the second housing part 330 has an end plate 332 that is configured to mate with the second end 314 of the first housing part 310. At a first end 331, the second housing part 330 has a first conduit section 334 which is a main conduit section of the second housing part 330. A second conduit section 336 branches off of the first conduit section 334 at a first location before the end plate 332. As shown, the second conduit section 336 can be formed at a right angle relative to the first conduit section 334 and is in direct fluid communication with the first conduit section 334. The first conduit section 334 extends to the end plate 332 at which an opening is formed to allow direct access to the first conduit section 334. In addition, a short second connector conduit 335 is provided and extends outwardly from the second conduit section 336 and runs parallel to the first conduit section 334. One end of the second connector conduit 335 is thus in fluid communication with the interior of the second conduit section 336. An opposite end of the second connector conduit 335 extends to the end plate 332 at which an opening is formed to allow direct access to the second connector conduit 335. With the end plate 332, the entrance to the second connector conduit 335 is thus directly above the entrance to the first conduit section 334.

It will be appreciated that shown, the sizes (diameters) of the various conduit sections described above can be different. The first connector conduit and the second connector conduit in one embodiment have the same dimensions (e.g., same diameter).

The HME 200 is designed to be held and contained within the end compartment 315. The HME 200 is designed so cover the open end of the first connector conduit 320 so that fluid flowing within the first connector conduit 320 is forced into contact with the HME 200. The size and shape of the HME 200 are thus selected in view of the size and shaped of the end compartment 315. As can be seen in the figures, in the illustrated embodiment, the HME 200 has a generally circular shaped body with a bottom cutout or notch in the form of a partial circle. In other words, the bottom edge of the HME 200 has an arcuate shaped notch that is sized and shaped to receive a curved top of the first conduit section 334. The HME 200 can thus nest and seat over the curved first conduit section 334.

Any number of traditional techniques can be used to attach the first housing part 310 to the second housing part 330. Since the HME 200 needs periodic replacement, the first and second housing parts 310, 330 are attached in a way that allows for separation thereof.

A first cap 351 can be provided for capping off the top end of the second conduit section 318 of the first part and a second cap 353 can be provided for capping of the top end of the second conduit section 336 of the second part. The caps 351, 353 are preferably removable.

A valve member 360 is provided and can be manipulated by a user to select one of a number of valve operating positions. The valve member 360 is configured to be disposed within the valve conduit section 318 and be rotatable therein. The valve member 360 has a cylindrical shaped body and includes an outer wall 362 on which indicia can be provided to indicate the different positions of the valve member 360. The cylindrical shaped body includes at least two openings that are formed circumferentially around the cylindrical shaped body. For example, there is a pair of openings that are formed 180 degrees apart.

The valve member 360 has two distinct positions or settings. A first valve setting is on in which the pair of first openings are aligned with the first conduit section 316. In this setting, only the first conduit section 316 is open (the second conduit section 318 is likewise open). Fluid entering the first conduit section 316 flows longitudinally (linear) within the first conduit section 316 to the first conduit section 334 of the second housing part 330. This position can be thought of as being an HME bypass valve position since the main flow path is along the first conduit sections 316. 334.

In a second position, the pair of openings are rotated 90 degrees so that neither of the openings are aligned with the first conduit section 316. In this position, flow through the first conduit section 316 is prevented and instead, all of the fluid flows into the second conduit section 318 and passes through the HME 200.

The rotation of the valve member 360 can be limited by a mechanism that allows for rotation of the valve only a certain number of degrees. For example, an inner edge of the valve member 360 that is inserted into the valve conduit 319 (valve cradle section) can have a circumferential notch formed therein. This notch extends a prescribed number of degrees. There is a complementary standoff (protrusion/tab) 175 that is formed in the valve cradle. The standoff 175 is received within the notch and the valve member 360 can only rotate a prescribed number of degrees. The degree of movement of the valve member 160 is between one position in which the standoff 175 contacts one end of the notch and another position in which the standoff 175 contact the other end of the notch. The arcuate length of the notch defines the degree of travel.

A first flow path is thus defined when the valve member 360 is in the first valve position. In the first flow path, the fluid enters the first conduit section 316 of the first housing part and flows linearly into the first conduit section 334 of the second housing part 330 and then can flow to the patient via a patient interface (e.g., mask or breathing tube) that is fluidly coupled to the second housing part 330.

In a second valve setting, the valve member 360 is positioned such that the openings in the valve member 360 are not aligned with the first conduit section 334. This position can be thought of as being an HME position all of the fluid entering into the first conduit section 316 is directed into the second conduit section 318 and passes through the HME 200.

A second flow path is thus defined when the valve member 360 is in the second valve position. In the second flow path, the fluid enters the first conduit section 316 of the first housing part and flows at a right angle into the second conduit section 318 and then flows into the first connector conduit 320. As the fluid flows along the first connector conduit 320, the fluid flows across the HME 200 and into the second connector conduit 335 before then flowing into the second conduit section 336 and then finally into the first conduit section 334 of the second housing part 330. In this setting, all of the fluid entering the first housing part is directed through the HME to provide HME treated fluid (breathing gas). Exhaled air can follow the reverse flow and go from the second housing part through the HME 200 to the first housing part.

During normal breathing and when no medication is to be delivered, the valve member 360 can be positioned in the second valve position and the fluid is directly exclusively along the second flow path and is conducted across the HME 200 and then subsequently is delivered to the patient. In the event that medication needs to be delivered to the patient, the valve member 360 is placed in the first valve position (HME bypass) and the medication enriched fluid bypasses the HME 200 and flows to the patient.

### HME Unit According to Third Embodiment

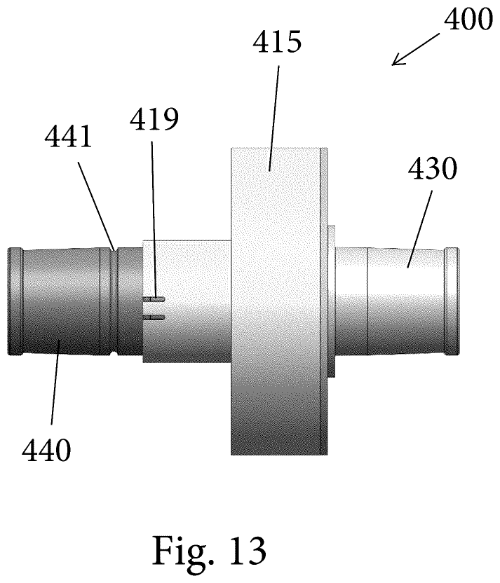

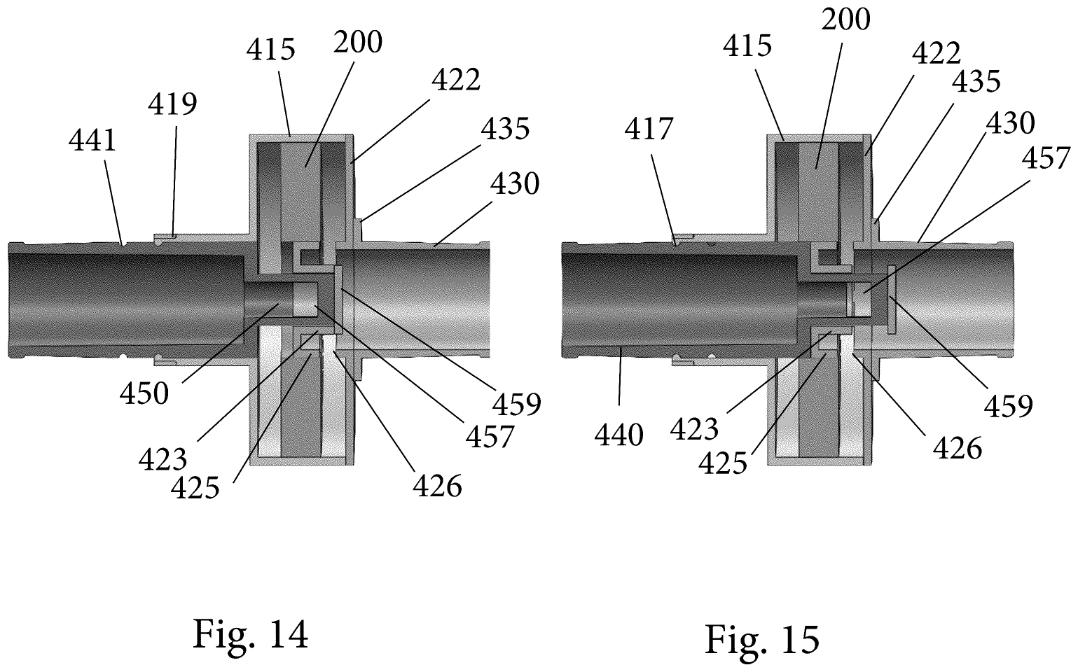

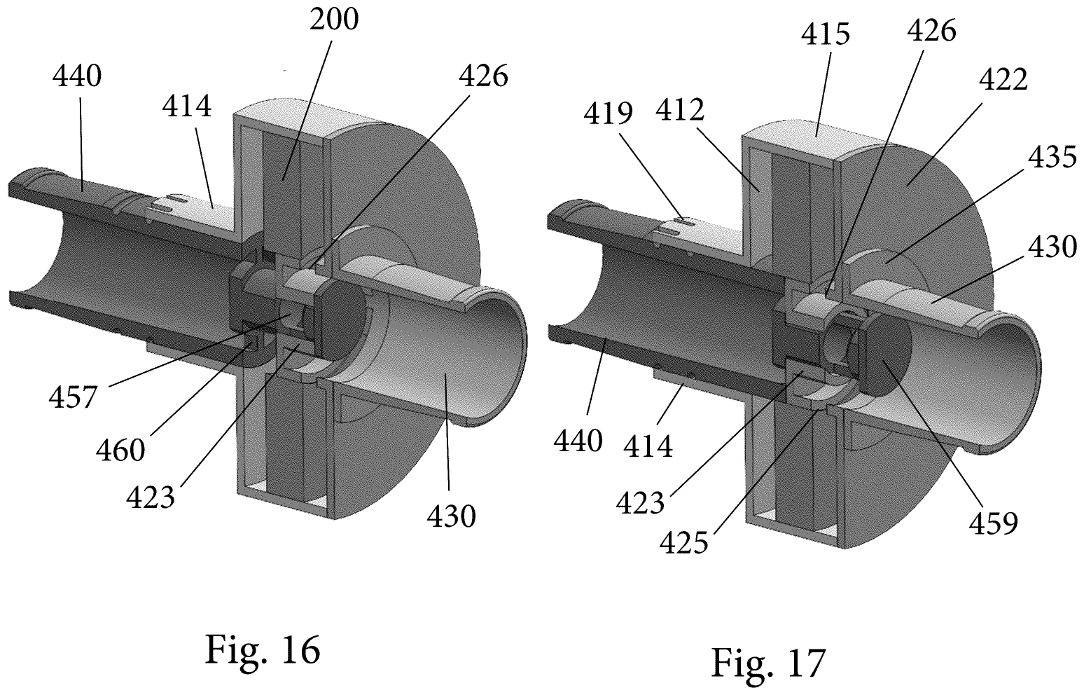

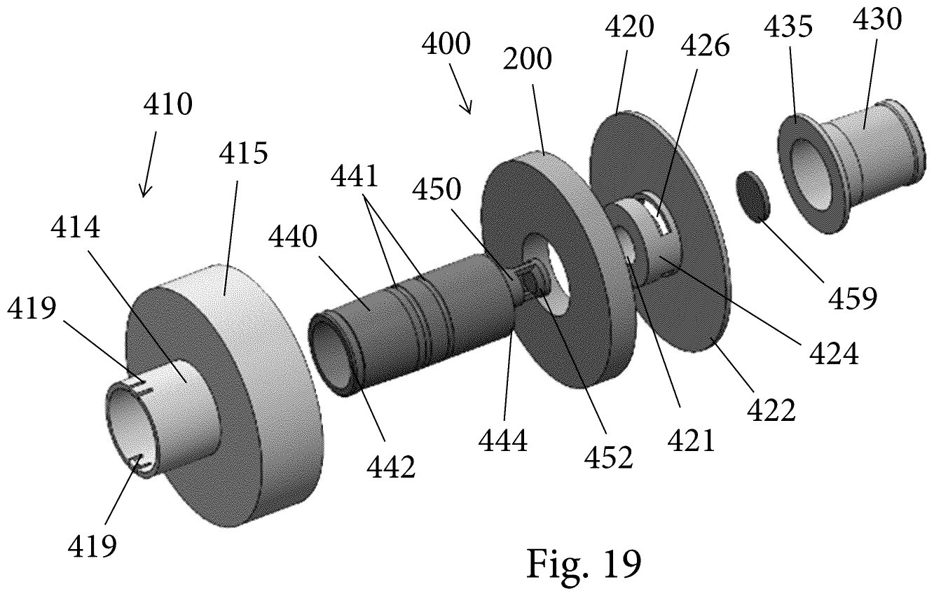

FIGS. 13-19 illustrate an HME unit 400 according to a third embodiment. The HME unit 400 includes a housing that is defined by a first housing part 410 that has an cylindrical shaped compartment 412 at one end and a first conduit section 414 that extends outwardly from the cylindrical shaped compartment 412 at the other end. The first conduit section 414 can be centrally located relative to the cylindrical shaped compartment 412 and is a tubular protrusion that can have a cylindrical shape. The hollow center of the first conduit section 414 directly opens into the cylindrical shaped compartment 412. The cylindrical shaped compartment 412 is defined by an outer side wall 415.

As shown, the first conduit section 414 can include notches or slots 419 formed therein to allow for flexing of the first conduit section 414. As described herein, the first conduit section 414 flexes to permit detachable locking with the nozzle 440 in at least two different preselected positions.

The HME unit 400 includes an end plate 420 that has an annular shaped outer flange 422 and a center hub 424 that extends outwardly from a first face of the flange 422. The flange 422 thus extends radially outward from the center hub 424. The center hub 424 can have a cylindrical shape and has a center through hole (bore) 421 that is defined by an inner wall 423 (e.g., an annular shape wall). The center hub 424 also has an outer wall 425 (e.g., annular shaped) that is disposed radially outward from the inner wall 423 and is directly attached to the outer flange 422. Along the outer wall 425, there are one or more slots 426. As illustrated, there can be two slots 426 each of which has an arcuate shape (semi-circular shape). As shown in the figures, when the end plate 420 seats against and is coupled to the outer side wall 415, the cylindrical shaped compartment 412 is closed off and defines the sealed space that receives the HME 200. In this embodiment, the HME 200, as shown, has an annular shape and is disposed within the cylindrical shaped compartment 412. The HME 200 seats against the outer wall 425 and preferably does not substantially cover the slots 426. In other words, the HME 200 preferably is located upstream of the slots 426 and a portion of the HME 200 can be located proximal to (upstream of) the proximal end wall of the center hub 424.

The HME unit 400 also includes a second conduit section 430 that seats against a second face of the flange 422. The second conduit section 430 is a hollow structure and includes an annular shaped flange 435 at one end. The annular shaped flange 435 seats against the second face of the flange 422. As shown, the second conduit section 430 can have a cylindrical shape. The diameter of the through hole (bore) of the second conduit section 430 is sized relative to the width (diameter) of the center hub 424 since, as described herein, fluid flows through these parts.

The HME unit 400 includes a nozzle 440 that is slidingly disposed within the first housing part 410. The nozzle 440 is a hollow part that has a main section that includes a first end 442 and an opposite second end 444. At the second end 444 of the main section, the nozzle 440 has a post 450 that extends outwardly from the second end 444. The post 450 is a hollow post that can have a cylindrical shape. The post 450 terminates in a distal end 452 that includes a seal member 459. The opposite proximal end of the post 450 is integrally formed with the second end 444. The seal member 459 has an enlarged shape relative to the post 450 and can be in the form of a flange. The seal member 459 can be securely attached to the distal end 452 of the post 450 using any number of techniques. The seal member 459 can have a disk shape. As shown in the figures, the size (diameter) of the seal member 459 is selected in view of the diameter of the inner wall 423 of the center hub 424 since in at least one operating position, the seal member 459 seats against the inner wall 423 so as to close off the center bore of the center hub 424.

The nozzle 440 includes along the exterior of its main section one or more first locking features 441. For example, the first locking features 441 can be in the form of a pair of grooves that are spaced apart. Each groove extends around the complete circumference of the main section of the nozzle 440. Each groove can have a concave shape as shown. The first locking features 441 are complementary to a second locking feature 417 that is part of the first conduit section 414. As shown, the second locking feature 417 can be in the form of a circular shaped inner protrusion that is formed inside of the first conduit section 414 at or near its free end. As described herein, the nozzle 440 is received within the hollow interior of the first conduit section 414 and travels axially therein. The second locking feature 417 travels along the exterior surface of the main section of the nozzle 440 until it is placed in registration with one of the first locking features 441 at which time, the second locking feature 417 snaps into place and engages the first locking feature 441. In other words, the circular shaped protrusion (lip) is received into the groove, thereby holding the nozzle 440 in a set location within the first conduit section 414. As described herein, the two grooves define two different locking positions of the nozzle 440.

Along the length of the post 450 there are one more openings 457 that are in fluid communication with the center bore of the post 450. As shown, the one or more nozzle openings 457 can consist of a pair of nozzle openings 457 that are formed along the side wall of the post 450 proximate to the distal end 452. These openings 457 allow fluid flowing through the main section of the nozzle 440 to flow into the center bore of the post 450 and then exit through the nozzle openings 457 when they are in the open position as discussed herein. The seal member 459 is thus located downstream of (distal to) the nozzle openings 457.

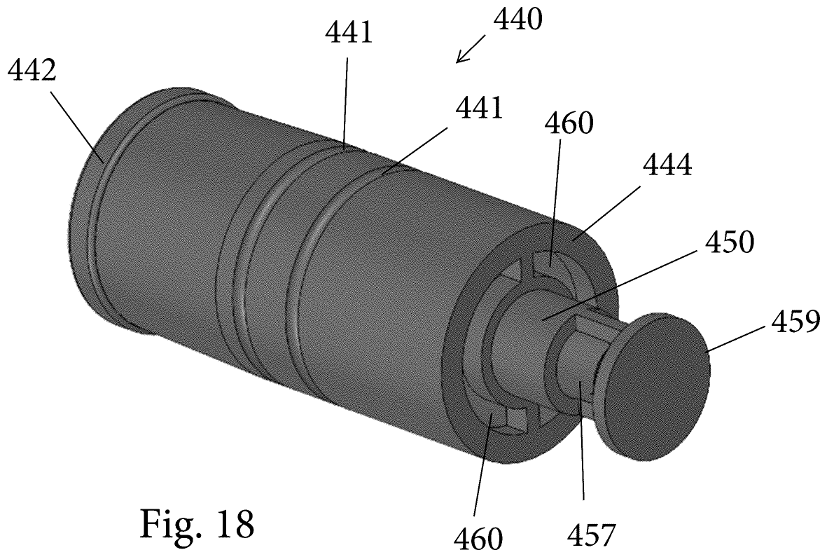

At the second end 444 of the main section, there are distal openings 460 that are formed circumferentially around the base of the post 450. The distal openings 460 are located radially outward of the post 450. As shown in FIG. 18, there can be two distal openings 460 each of which has a semi-circular (arcuate) shape. Between the two distal openings 460 there can be two separating ribs. The two distal openings 460 are in fluid communication with the hollow interior of the main section of the nozzle 440 and thus, when the distal openings 460 are in an open position, fluid flowing within the hollow interior of the main section can exit through the distal openings 460 and flow radially around the post 450.

The HME unit 400 operates in a push/pull manner in that the nozzle 440 is designed to be axially displaceable in a push/pull manner. A first operating position of the HME unit 400 is one in which all of the fluid entering into the HME unit 400 is directed through the HME 200. In this first operating position, the nozzle 440 is in a first position in which the second locking feature 417 (male protrusion) engages the second locking feature 441 (groove) that is closest to the post 450. In this position, the seal member 459 seats against the inner wall 423 so as to close off the center bore of the center hub 424. In addition, the nozzle openings 457 are closed due to them being disposed against the inner wall 423 of the center hub 424. In this position, fluid cannot exit through the nozzle openings 457. However, the distal openings 460 are open as shown in the figures and provide a direct flow path into the cylindrical shaped compartment that houses the HME 200. Fluid thus exits the distal openings 460 and flows through the HME 200 before then flowing into the slots 426 that are formed in the outer wall 425. These slots 426 are open and in fluid communication with the hollow center of the second conduit section 430 which leads to a patient interface and to the patient. As one can appreciate in this first operating position, the fluid (e.g., breathing gas) flows through the hollow interior of the nozzle 440 and then exits through the distal openings 460 and is conduced across the HME 200 before then flowing into the hollow center of the second conduit section 430.

This first operating position is one in which the HME 200 is online and all of the incoming fluid (breathing gas) is directed across the HME 200. This first operating position is suitable for normal breathing but not suitable for when medication or the like must be delivered.

In the event that medication or the like must be delivered to the patient, the HME unit 400 is placed in its second operating position by axially moving the nozzle 440 until the second locking feature 417 (male protrusion) engages the second locking feature 417 (groove) that is furthest from the post 450. In this position, the seal member 459 does not seats against the inner wall 423 but rather is spaced therefrom. As a result, the nozzle openings 457 are open since they are now spaced from the inner wall 423 of the center hub 424. In other words, the inner wall 423 does not cover the nozzle openings 457. In contrast, the distal openings 460 are closed since they are seated against the end wall of the center hub 424. In addition, the cylindrical shaped compartment is likewise closed off and access to the HME 200 is prevented. Instead, the fluid flowing into the main section of the nozzle 440 flows into the hollow center of the post 450 and exits through the open nozzle openings 457 into the hollow interior of the second conduit section 430. The HME 200 is thus bypassed and this operating position is suitable for when medication or the like needs to be delivered to the patient.

The nozzle 440 is thus part of a push/pull arrangement in which the user grasps the exposed end of the nozzle 440 and either pulls or pushes it relative to the assembled part of the HME unit 400 including the first housing part 410.

### HME Unit According to Fourth Embodiment

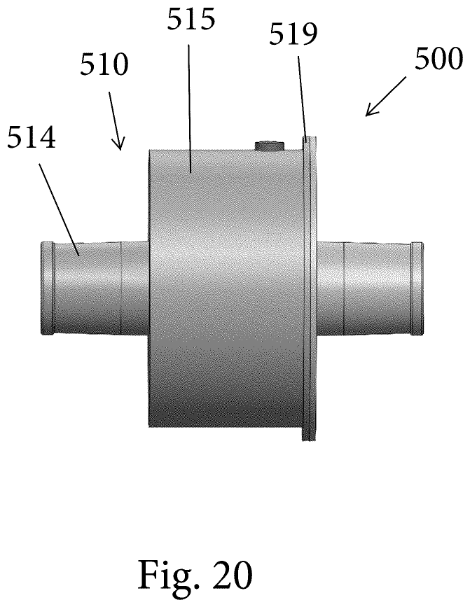

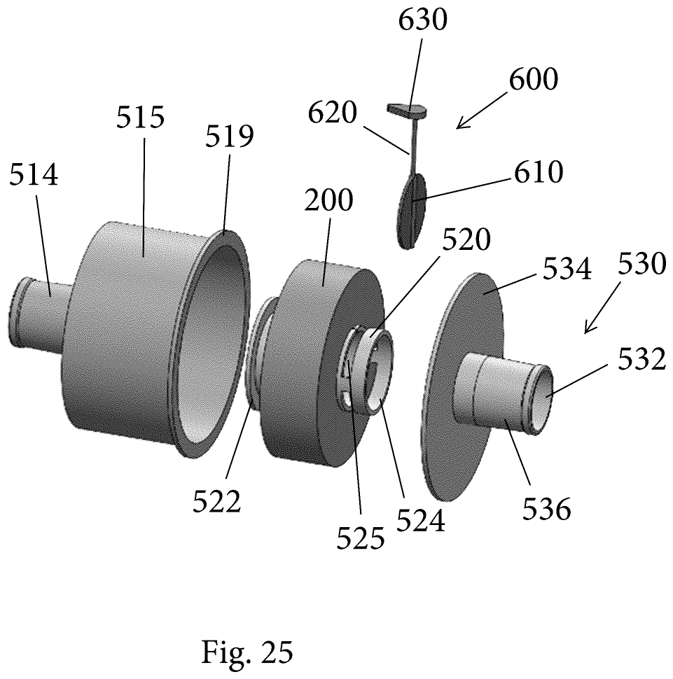

FIGS. 20-25 illustrate an HME unit 500 according to a fourth embodiment. The HME unit 500 includes a housing that is defined by a first housing part 510 that has a cylindrical shaped compartment 512 at one end and a first conduit section 514 that extends outwardly from the cylindrical shaped compartment 512 at the other end. The first conduit section 514 can be centrally located relative to the cylindrical shaped compartment 512 and is a tubular protrusion that can have a cylindrical shape. The hollow center of the first conduit section 514 directly opens into the cylindrical shaped compartment 512. The cylindrical shaped compartment 512 is defined by an outer side wall 515. A lip 519 is formed at one end of the outer side wall 515.

The HME unit 500 further includes an inner core 520 that is configured to be inserted into the cylindrical shaped compartment 512. One end of the inner core 520 can be coupled to the end wall of the cylindrical shaped compartment 512. For example, the end of the inner core 520 can contain a flange (lip) that seats against the inner surface of the end wall of the compartment 512. The inner core 520 can be considered to be an insert that is disposed in the cylindrical shaped compartment 512. The inner core 520 has a cylindrical shape with a first end 522 and an opposite second end 524. The inner core 520 includes one or more first openings 523 formed along its length and includes one or more second openings 525 formed along its length. As illustrated, the one or more first openings 523 are located proximate the first end 522 and the one or more second openings 525 are located proximate the second end 524.

In the illustrated embodiment, there are two first openings 523 that are separated from one another and are formed circumferentially about the inner core 520. For example, the two first openings 523 can be in the form of two arcuate slots that are located opposite one another. The two first openings 523 preferably extend greater than 300 degrees about the inner core 520 and can be more than 330 degrees, etc. Similarly, in the illustrated embodiment, there are two second openings 525 that are separated from one another and are formed circumferentially about the inner core 520. For example, the two second openings 525 can be in the form of two arcuate slots that are located opposite one another. The two second openings 525 preferably extend greater than 300 degrees about the inner core 520 and can be more than 330 degrees, etc. Between the two first openings 523 and the two second openings 525, there is a smooth center region that is free of openings. The HME 200 is for placement about this smooth center region such that the HME 200 seats against the outer surface of the inner core 520. The HME 200 has an annular shape and extends circumferentially about the inner core 520. The outer diameter of the HME 200 is also selected so that it seats against an inner surface of the outer side wall 515. The width of the HME 200 is thus the same as or close to a length of the smooth center region of the inner core 520.

The HME unit 500 also includes an end plate 530. The end plate 530 has an open first end 532 and an opposite second end at which an enlarged flange 534 is formed. The end plate 530 also includes a second conduit section 536 that extends outwardly from a first face of the enlarged flange 534 and has a tubular shape. The second conduit section terminates at one end with the open first end 532. The enlarged flange 534 thus extends radially outward from the second conduit section 536 and has a center opening that leads into the hollow center of the second conduit section 536. The enlarged flange 534 is designed to seat against and be sealed to the lip 519 as a means for closing off the cylindrical shaped compartment 512. Any number of techniques can be used to seal the enlarged flange 534 to the lip 519. When the enlarged flange 534 seats against the lip 519, both the first conduit section 514 and the second conduit section 536 are in fluid communication with the hollow interior of the cylindrical shaped compartment 512.

The HME unit 500 also includes a movable (rotatable) valve 600 that is configured to control the flow of fluid (e.g., breathing gas) through the HME unit 500. The valve 600 is in form of a disk 610 that is connected to a post 620 at a bottom end of the post 620. The post 620 can attach to a center of the disk 610. At the top end of the post 620 there is an actuator 630 that is accessible to the user and permits the user to move the valve 600 between a fully opened position and a fully closed position as well as intermediate valve locations therebetween.

The outer side wall 515 has an opening through which the post 620 passes so as to position the actuator 630 along the exterior of the outer side wall 515. The actuator 630 can have any number of different shapes including a triangular shape as shown. The outer side wall 515 preferably includes indicia that indicates the different valve positions. To position the valve 600 in one of the settings, the user twists (rotates) the actuator 630 and in the event of a triangular shaped actuator 630, the point of the actuator 630 is pointed toward the indicia that identifies the intended, desired valve position. For example, if it is desired to position the valve 600 in a fully opened position, the user rotates the actuator 630 such that the point of the actuator 630 points to the fully opened indicia.

As shown in the figures, the valve 600 is located between the one or more first openings 523 and the one or more second openings 525 and is positioned at one end (along one end face) of the HME 200. The disk 610 is received within the hollow center of the inner core and has a complementary shape. In particular, the disk 610 has a diameter than is equal to the inner diameter of the inner core and therefore, the disk 610 is capable of sealing against the inner surface of the inner core so as to completely occlude the inner space (bore) of the inner core.

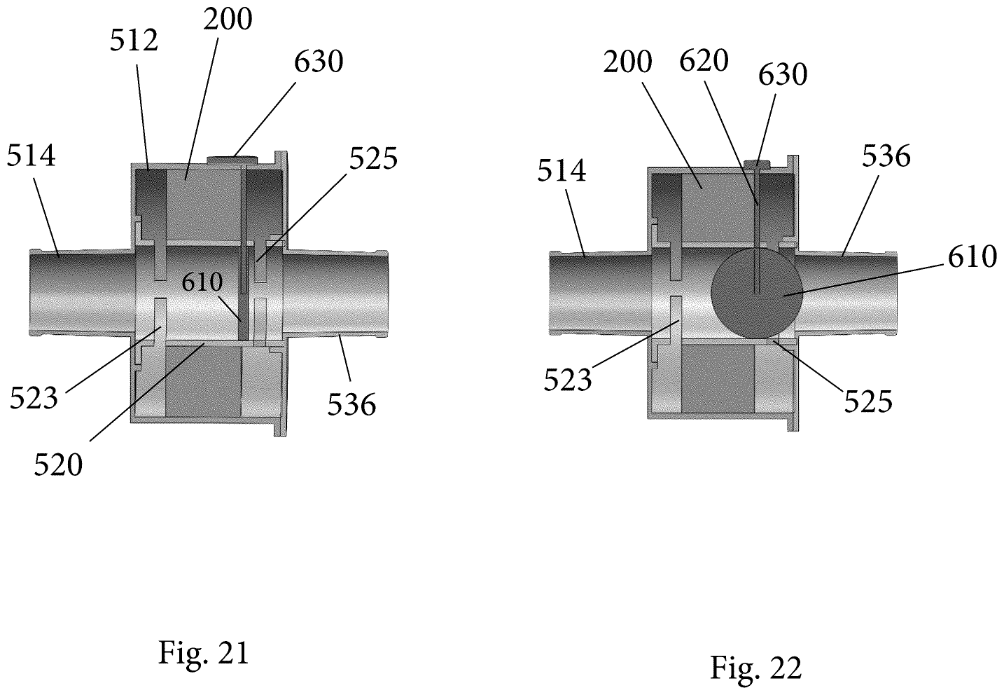

FIGS. 21 and 24 illustrate the valve 600 in the closed position. In the closed position, the valve 600 is positioned such that the disk 610 thereof is located in a plane that is perpendicular to a longitudinal axis of the inner core. In other words, the disk 610 completely occludes the inner bore of the inner core as shown.

The closed valve position causes the breathing gas (e.g., oxygen) to be diverted to the HME 200. In other words, when the valve 600 occludes the inner bore of the inner core, the breathing gas flows through the first openings 523 into the cylindrical shaped compartment 512 at a location upstream of the HME 200. The breathing gas flows through the HME 200 and then flows through the one or more second openings 525 formed in the inner core into the hollow inside of the inner core. Since the first and second openings 523, 525 are located on opposite sides of both the valve 600 and the HME 200, breathing gas effectively flows from the inner core to the cylindrical shaped compartment 512, across the HME 200, and then flows back into the inner core. This closed position of the valve 600 is thus the normal operating position of the HME unit 500 when it is desired for all of the delivered breathing gas to flow through the HME 200.

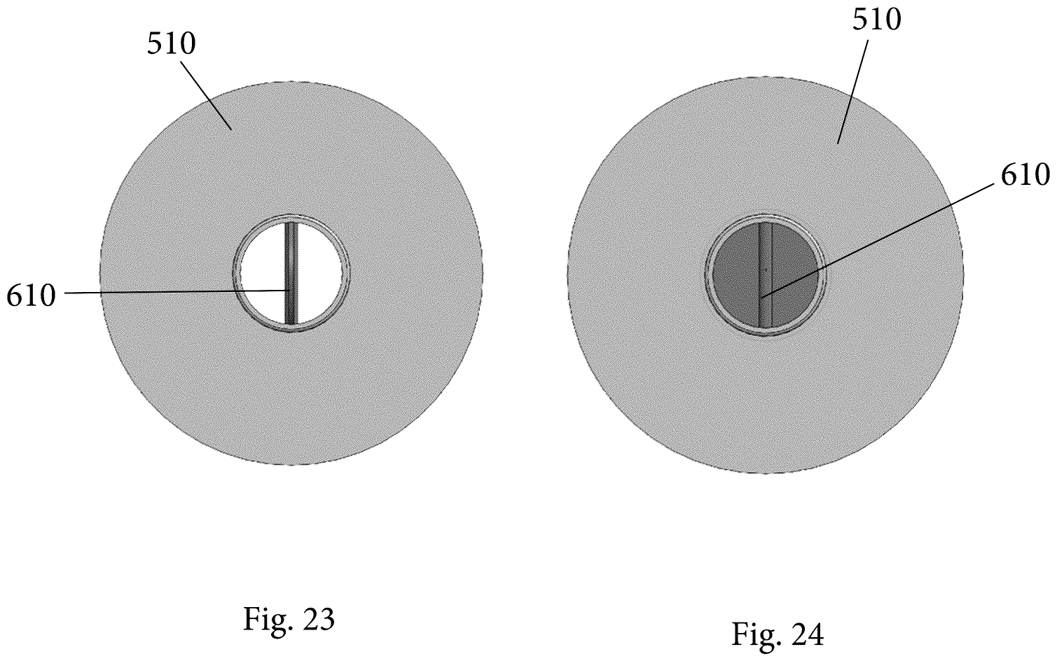

FIGS. 22 and 23 show the valve 600 in the open position. In the open position, the valve 600 is positioned such that the disk 610 is located in a plane that is parallel to and/or coaxial with the longitudinal axis of the inner core. In this orientation, as shown in FIG. 23, the disk 610 partitions (bifurcates) the inside of the inner core into two spaces through which fluid can flow. In this open position, the breathing gas can flows axially through the inner core. While the cylindrical shaped compartment 512 remains open, fluid dynamics dictate that most if not all of the breathing gas flows axially within the inner core since this is the path of least resistance.

It will also be appreciated that it is possible that the valve 600 can be placed in a position that is between the fully open position and the fully closed position. In these intermediate positions, the axial flow of the breathing gas can be increasingly impeded which causes more gas to flow through the HME 200.

### Heat Moisture Exchanger

A heat and moisture exchanger (HME) 200 is provided and is disposed within the housing 110 and is within the supplemental gas flow pathway through the housing 100.

As is known, a heat and moisture exchanger is a device that is used in mechanically ventilated patients intended to help prevent complications due to drying of the respiratory mucosa, such as mucus plugging and endotracheal tube (ETT) occlusion. The HME 200 is typically formed of a layer of foam or paper embedded with a hydroscopic salt, such as calcium chloride.

### HME Unit According to Fifth Embodiment

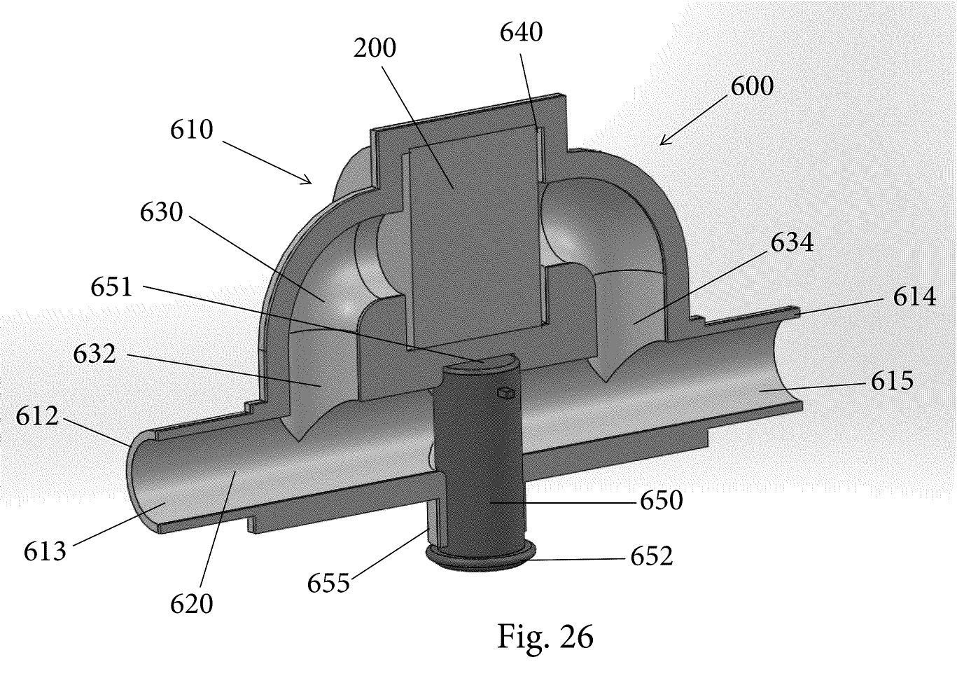

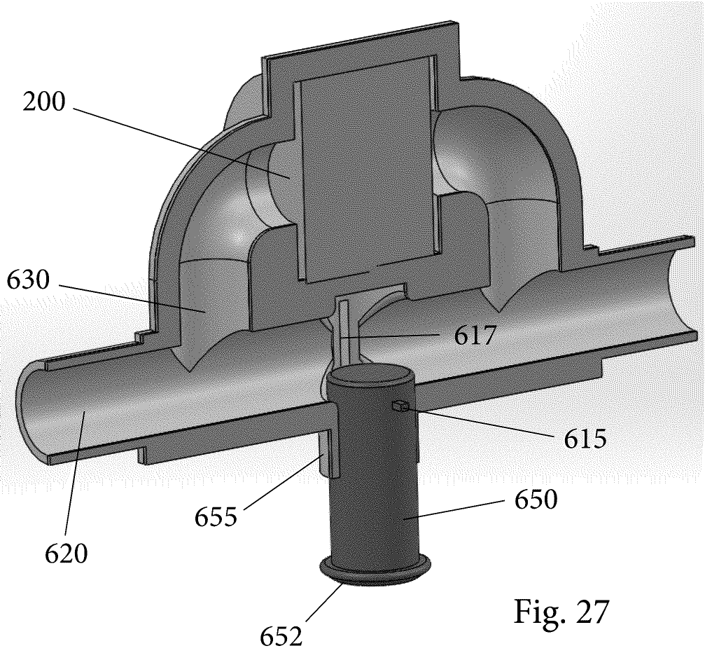

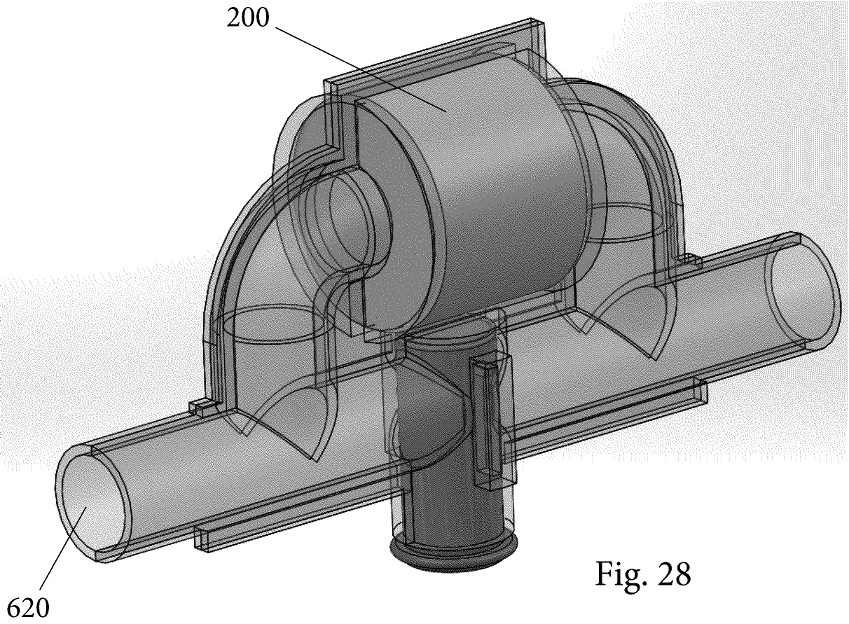

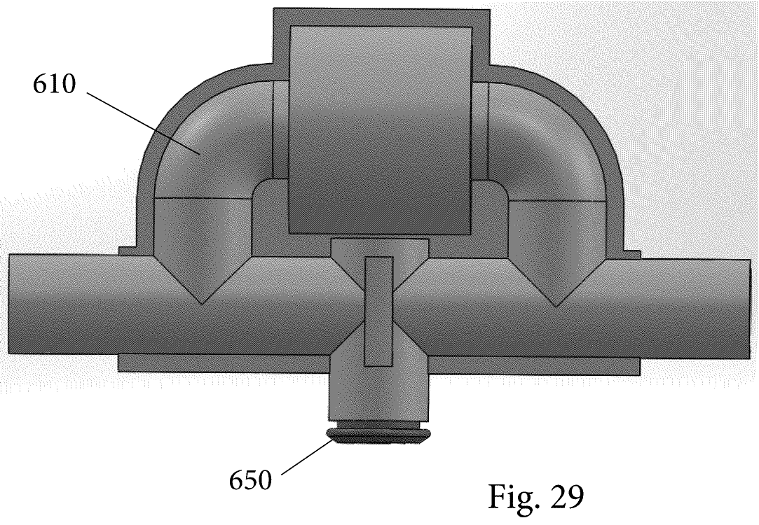

FIGS. 26-29 illustrate an HME unit 600 according to a fifth embodiment. The HME unit 600 includes a housing 610 that includes a number of open ports. The housing 610 has a first end 612 and an opposing second end 614. The first end 612 can be attached to a source of fluid, while the second end 614 can be attached to a patient interface such as a breathing mask or breathing tube. In the illustrated embodiment, the fluid thus flows from left to right; however, the symmetric nature of the HME unit 600 permits it to be oriented differently.

At the first end 612, there is a first end port 613 and at the second end 614, there is a second end port 615.

While, the housing 610 is described as being a single part, it will be appreciated that it can consist of two or more parts that are assembled. For example, the housing 610 can be formed of two parts and thus resemble a clamshell. This type of construction allows for insertion of other parts into the interior of the housing 610 and then the two parts are assembled around the inserted parts.

Within the HME unit 600 there are two flow paths defined by conduits formed therein as described below. In particular, the HME unit includes a main flow path generally identified at 620 that is formed axial to the first end port 613 and the second end port 615. Thus, in the illustrated embodiment, the main flow path is a linear flow path from the first end 612 to the second end 614.

The housing 610 also includes an HME bypass flow path 630 that has a first end 632 and an opposite second end 634. Both the first end 632 and the second end 634 are in fluid communication with the main flow path. The first end 632 connects to the main flow path at a first location and the second end 634 connects to the main flow path 620 at a second location spaced from the first location along the main flow path. In the illustrated embodiment, the HME bypass flow path 630 has an upside down U-shape with the two legs fluidly connected to the main flow path at the first and second locations.

Along the HME bypass flow path 630, there is an HME cavity 640 which has dimensions greater than the dimensions of the HME bypass flow path 630; however, the HME bypass flow path 630 is in fluid communication with both ends of the HME cavity 640 such that fluid flowing within the HME bypass flow path 630 flows into the expanded HME cavity 640. The HME cavity 640 holds the HME 200 and therefore, when fluid flows along the HME bypass flow path 630, the fluid flows into the HME cavity 640 and more particularly, flows through the HME 200 before then returning to the main flow path at the second location.

As shown, the HME bypass flow path 630 has no valves along its length and therefore, it always is open in that fluid is always permitted to flow the length of the HME bypass flow path 630.

The HME unit 600 also includes a valve member 650 that moves between a first position and a second position and more particularly, the first position is a fully open position in which the main flow path 620 is fully open and the second position is a fully closed position in which the main flow path 620 is completely closed (completely occluded).

The valve member 650 is in the form of a movable plug that is configured to move axially between the first and second positions. The valve member 650 is located within a valve port 655 formed as part of the housing 610. The valve port 655 is located between the first and second locations of the main flow path. The valve port 655 directly communicated with and opens directly into the main flow path. This arrangement allows the valve member 650 to move into and out of the main flow path.

The valve member 650 can, as shown, have an enlarged head 652 that allows the valve member 650 to be grasped and manipulated and moved between the first and second positions. The illustrated valve member 650 is sealingly disposed within the valve port 655 and can be in the form of a solid cylinder, such of a material such as rubber or plastic. The enlarged head 652 can be a flange that extends radially outward from one end of the cylinder shaped main body of the valve member 650.

The valve member 650 can slide within the valve port 655 and enter into the main flow path. Thus, one end portion opposite the enlarged head 652 can move into the main flow path. The degree to which this one end portion is contained within the main flow path determines the degree to which the main flow path is open. In the first, fully open position, the first end portion of the valve member 650 does not intrude into the main flow path and thus, fluid (gas) can freely flow within the main flow path from the first end port 613 to the second end port 615. In contrast, in the second, closed position, the first end portion of the valve member 650 completely occludes the main flow path and prevents any fluid from flowing within the main flow path from the first end to the second end.

The valve member 650 thus moves axially and can represent a plug that plugs the main flow path when the valve member 650 is pushed inward in a direction toward the HME 200. Inside the housing there can be a stop 651 that alerts the users that the valve member 650 is fully inserted and thus, the main flow path is completely closed off. The stop 651 can be in the form of a recessed area formed along the top of the inner conduit that defines the main flow path. In addition, the user can be alerted that the valve member 650 is in the fully closed position when the enlarged head 652 contacts and seats against the exposed end of the valve port 655. In other words, the valve member 650 can be pushed fully inward to achieve its fully closed position.

In addition, the valve member 650 can include a guide feature that permits the valve member 650 to slide in an axial manner within the housing 610. In particular, a pin in groove (slot) arrangement can be provided. The pin can be one or more pins 615 that are formed along the cylindrical shaped body of the valve member 650 and the groove can be one or more grooves 617 formed within the housing.

The cylindrical shaped body of the valve member 650 can include a visual marking that indicates to the user when the valve member 650 is in the fully open position. For example, a line that extends around the circumference can be formed on the cylindrical shaped body and there can be text that indicates that the valve member 650 is in the fully open position. In this type of indicator, when the user can see the marking as the valve member 650 is pulled outward, the user knows the fully open position has been achieved. The marking becomes visible when the line is exposed below the edge (end) of the valve port 655 when the valve member 650 is pulled out. The user can thus pull out the valve member 650 until at least the marking is visible.

When the valve member 650 is in the fully closed position, the main flow path 620 is completely closed and this diverts fluid entering the first end port 613 to flow into the first end 632 of the HME bypass flow path 630. Since all of the incoming fluid is directed into the HME bypass flow path 630, this fluid must flow through the HME 200 before it enters back into the main flow path at the second location and then flows to the patient.

### HME Unit According to Sixth Embodiment

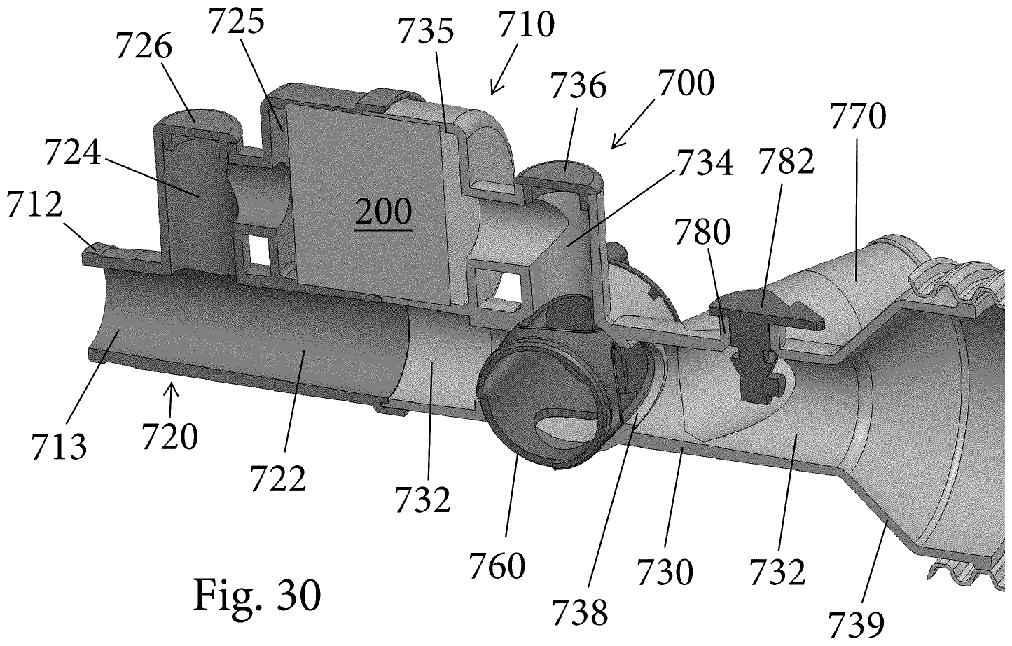

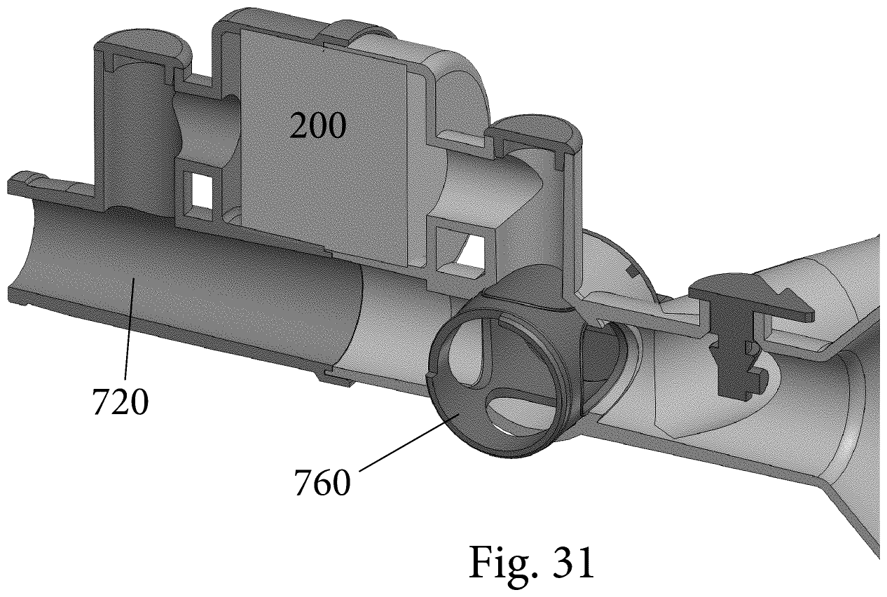

FIGS. 30-33 illustrate an HME unit 700 according to a sixth embodiment. The HME unit 700 includes a housing 710 that includes a number of open ports. The housing 710 generally has a first end 712 and an opposing second end 714. The second end 714 can be attached to a source of fluid, while the first end 712 can be attached to a patient interface such as a breathing mask or breathing tube. In the illustrated embodiment, the fluid thus flows from left to right; however, the symmetric nature of the HME unit 700 permits it to be oriented differently.

At the first end 712, there is a first end port 713 and at the second end 714, there is a second end port 715.

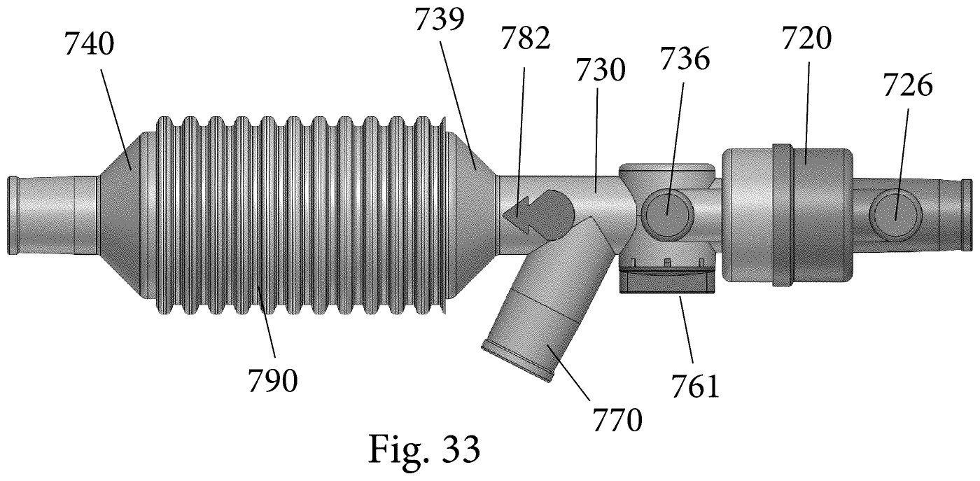

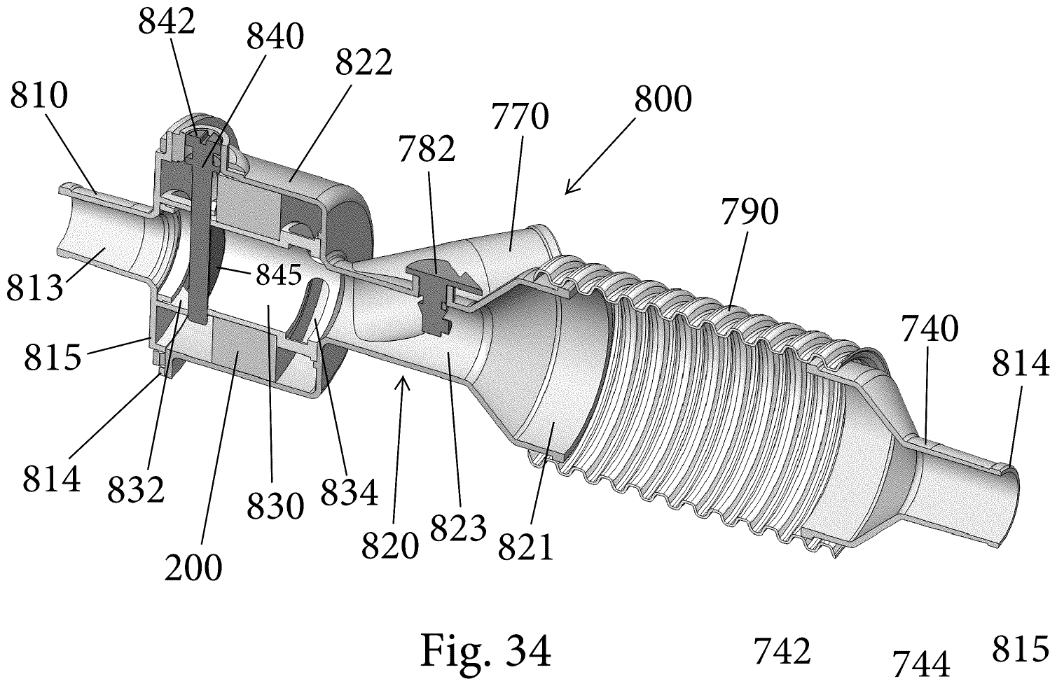

The housing 710 is formed of a number of individual parts that are assembled together as best shown in FIGS. 33 and 34. For example, the housing 710 can include a first part 720, a second part 730 and a third part 740.

The first part 720 defines the first end 712 and can be a molded structure that includes a first main conduit 722 that at a first end thereof defines the first end port 713. The first main conduit 722 can be linear in nature. The first part 720 also defines an HME bypass flow path and includes a first HME bypass section 724 that is in fluid communication with the first main conduit 722 at a first location. The first HME bypass section 724 can include a first enlarged HME cavity 725 that is along the HME bypass flow path.

The first HME bypass section 724 can also include a first plug port 726 that can receive a first plug 727 that seals the first plug port 726.

The second part 730 is configured to mate with the first part and in particular, a friction fit or snap-fit or other type of connection can be made to connect the two parts. The second part 730 has a similar shape as the first part 720 and in certain aspects can be considered to have a mirror image as the first part 720. The second part 730 can be a molded structure that includes a second main conduit 732 that is coaxial with and thus in fluid communication with the first main conduit 722. The second main conduit 732 is thus linear in nature. The second part 730 also defines an HME bypass flow path and includes a second HME bypass section 734 that is in fluid communication with the second main conduit 732 at a second location that is upstream of the first location. The second HME bypass section 734 can include a second enlarged HME cavity 735 that is along the HME bypass flow path. The combined first and second enlarged HME cavities 725, 735 define the space that receives the HME 200. As in the other embodiments, the HME 200 is located within the HME bypass flow path and thus, fluid that is diverted into the second HME bypass section 734 flows through the HME 200 and then into the first HME bypass section 724 before then flowing back to the main flow path.

The second HME bypass section 734 can also include a second plug port 736 that can receive a second plug 737 that seals the second plug port 736.

The second part 730 also includes a valve compartment 738 that is located at the second location which is the intersection between the second HME bypass section 734 and the second main conduit 732. The valve compartment 738 can have enlarged dimensions compared to each of the second HME bypass section 734 and the second main conduit 732. With the valve compartment 738 a valve member 760 is located. The valve member 760 is rotatable within the valve compartment 738 and can include an exterior section 761 that is accessible to the user to permit the user to grasp and rotate the valve member 760. The valve member 760 has three openings formed therein with two being opposite one another and the third being offset 90 degrees from the others. In the fully open position of the two openings are aligned with the second main conduit to permit free flow through the second main conduit. Third opening can be aligned with the second HME bypass section 734 or it can be offset and not aligned therewithin. Even if the third opening is aligned with the second HME bypass section 734, flow resistance through the HME bypass flow path is much greater due to the presence of the HME 200 and therefore fluid flows through the main conduit and is not diverted to the HME bypass flow path (by flowing into the second HME bypass section 734). To close the main flow path, the valve member 760 is rotated so that the two opposite openings are not axially aligned with the main flow path.

The second part 730 has an exhalation side port 770. The exhalation side port 770 extends radially outward and provides a path for exhaled air to be exhausted.

The second part 730 also includes an MDI access port 780 that is in fluid communication with the main conduit. A cap 782 is sealingly inserted into the MDI access port 780. The cap 782 can have a center lumen that is formed through the cap 782 and leads to and is in fluid communication with the main flow path. The cap 782 can be of a type that has an outer hinged portion 784 that can be lifted up to expose the center lumen. An MDI device can then be mated to the center lumen so that when the MDI device is operated, the expelled medication from the MDI device is injected into the main flow path. The MDI access port 780 is located upstream from the exhalation side port 770.

The second part 730 also includes an enlarged end portion 739 that is upstream of the MDI access port 780.

An expandable/collapsible corrugated tubing 790 is coupled at one end to the enlarged end portion 739. In the expanded state, the interior space (volume) within the tubing 790 is increased, while in the collapsed state, the interior space (volume) within the tubing 790 is reduced.

The third part 740 which is intended to be fluidly connected to a ventilator circuit is coupled to the other end of the tubing 790 and thus the tubing 790 is located between the second and third parts 730, 740. The third part 740 has an enlarged end portion 742 that is coupled to the tubing 790 and has a smaller main body 744 that is coupled to the ventilator circuit.

As with the other embodiments, the HME bypass flow path is configured to force all of the fluid (breathing gas) through the HME 200 when the user selects this mode of operation as by manipulating (turning) the valve member.

When the other operating mode is selected in which the main flow path is open and represents that primary flow path, the breathing gas flows primarily through the main flow path since even if the HME bypass flow path remains open (which it always is), the flow resistance of the HME 200 and the natural resistance of the torturous HME bypass flow path causes essentially all of the breathing gas to flow axially (linearly) within the main flow path.

It will be appreciated that when the MDI device is used, the valve member 760 is open to allow the injected medication to flow through the main flow path to the patient.

### HME Unit According to Seventh Embodiment

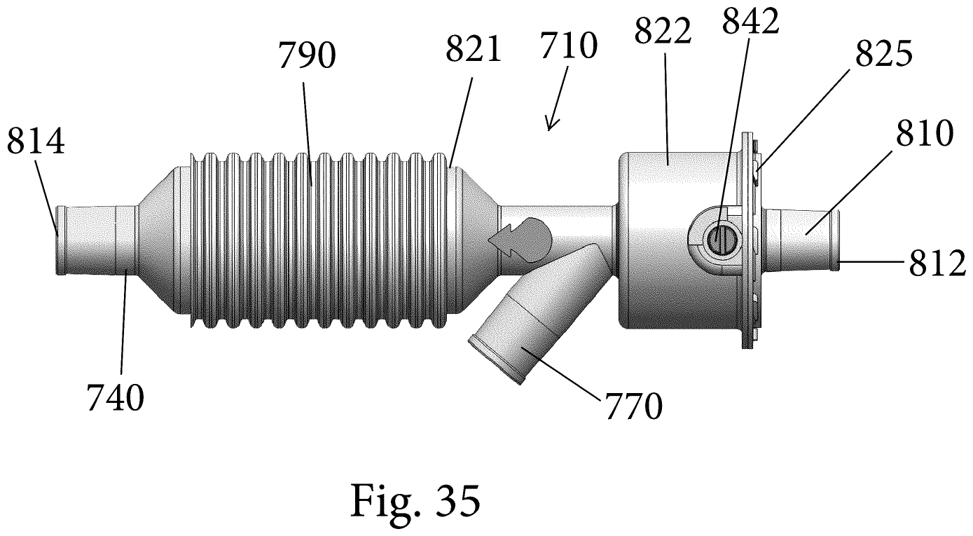

FIGS. 34-37 illustrate an HME unit 800 according to a seventh embodiment which is similar to the HME unit 700. Due to the similarities, like elements are numbered alike with respect to these two embodiments.

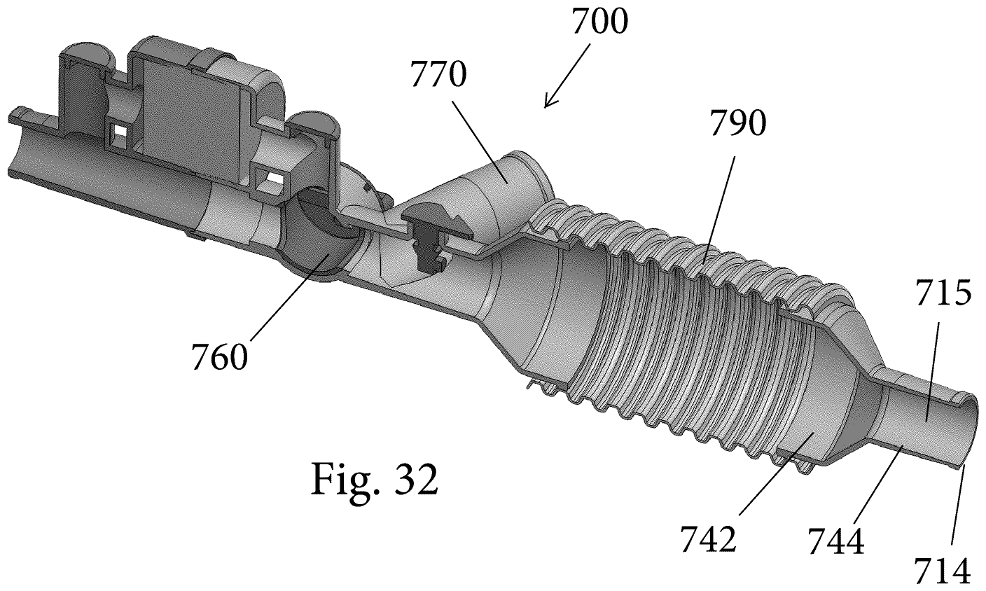

The HME unit 800 includes a housing that includes a number of open ports. The housing 810 generally has a first end 812 and an opposing second end 814. The second end 814 can be attached to a source of fluid, while the first end 812 can be attached to a patient interface such as a breathing mask or breathing tube. In the illustrated embodiment, the fluid thus flows from right to left in FIG. 32.

At the first end 812, there is a first end port 813 and at the second end 814, there is a second end port 815.

The housing is formed of a number of individual parts that are assembled together as best shown in FIG. 34. For example, the housing can include a first part 810, a second part 820 and the third part 740.

The first part 810 defines the first end 812 and can be a molded structure that includes a first main conduit 811 that at a first end thereof defines the first end port 813. The first main conduit 811 can be linear in nature. The first part 810 includes a flange 815 that includes a plurality of openings formed circumferentially about the flange 815. The flange 815 extends radially outward and surrounds the main tubular portion of the first part 810. The first part 810 includes another tubular section 819 that is on the other side of the flange 815. The other tubular section 819 can have a diameter that is greater than a diameter of the main tubular portion located on the other side of the flange 815.

The second part 820 includes an enlarged first section 822 that has a hollow interior, an intermediate tubular section 823 and an enlarged second section 821. The intermediate tubular section 823 that is located between the two enlarged sections 822, 821. The diameter of the intermediate tubular section 823 is less than the diameters of the two enlarged sections 822, 821.

The second part 820 includes the exhalation side port 770 that extends radially outward and provides a path for exhaled air to be exhausted. The second part 820 includes a plurality of locking tabs 817 that are received within the plurality of openings formed circumferentially about the flange 815 to couple the first part 810 and the second part 820 in a sealed manner.

The cap 782 and the MDI access port 780 are also shown and form part of the second part 820.

An inner tubular member 830 is provided and is disposed and anchored within the enlarged first section 822 of the second part 820. The inner tubular member 830 has a first opening (slot) 832 formed therein proximate a first end and a second opening (slot) 834 proximate a second end. The openings 832, 834 can have an arcuate shape due to the tubular nature of the inner tubular member 830. Around the inner tubular member 830, the HME 200 is present. The HME 200 can thus has an annular shape and is disposed within the hollow interior of the enlarged first section 822 and seats against the inner tubular member 830 that passes through the center hole of the HME 200. The inner tubular member 830 can include outer circumferential flanges that guide and locate placement of the HME 200 about the inner tubular member 830. As shown, the HME 200 is located between the openings 832, 834.

A movable valve member 840 is provided and passes into the enlarged first section 822 and through the inner tubular member 830. The valve member 840 includes an exterior head 842 that is located along the exterior of the second part and can be manipulated by the user. As shown, the head 842 can include a slit that can receive a tool (screwdriver like) and permit rotation of the valve member 840. The valve member 840 is located between the openings 832, 834.

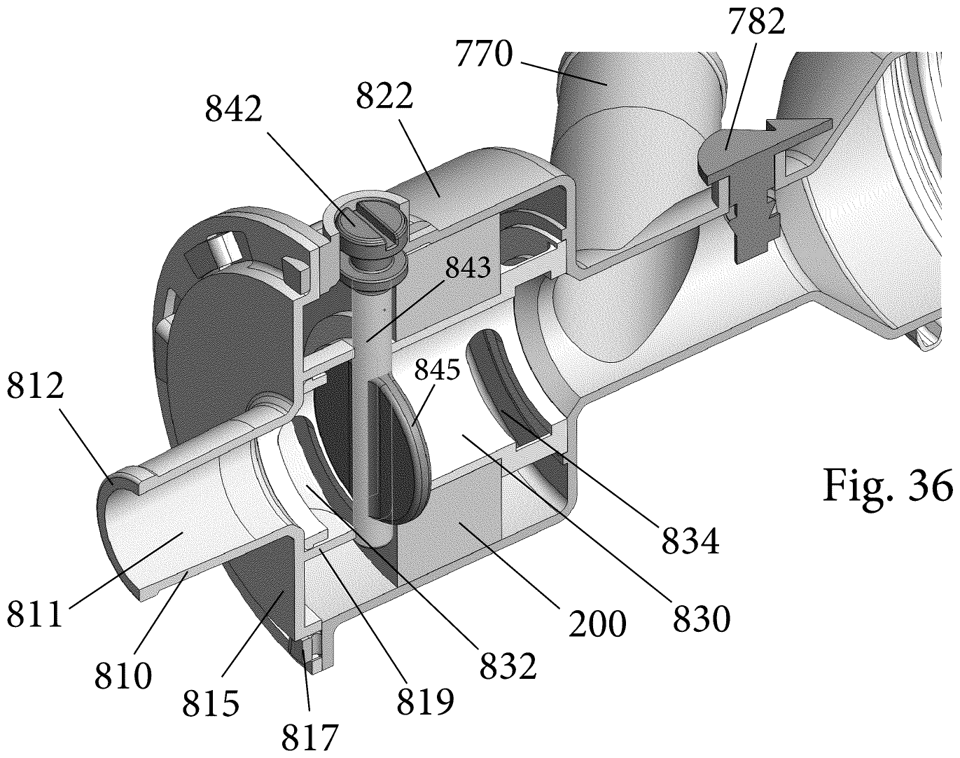

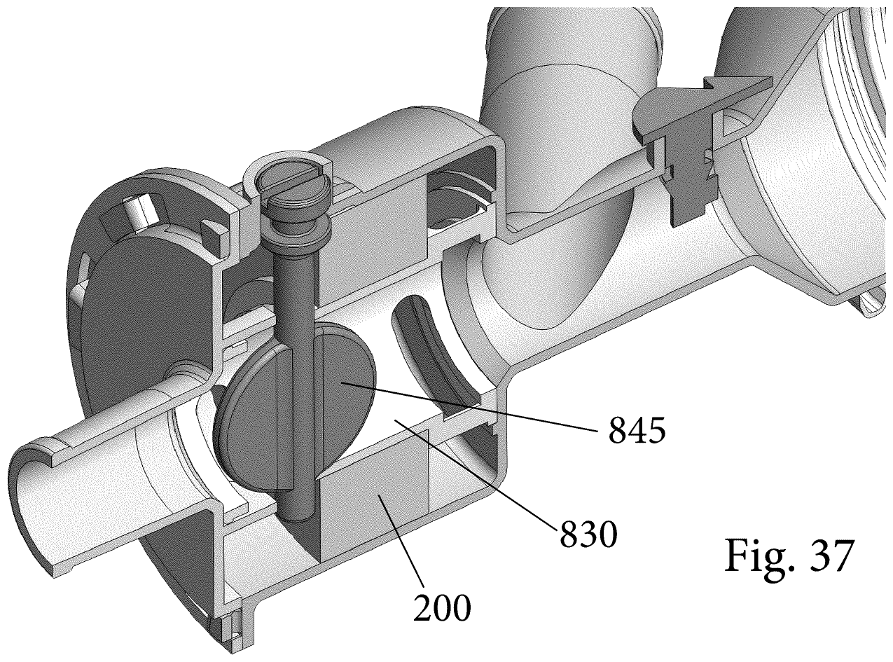

The valve member 840 can have a post 843 with the head 842 at one end. The other end of the post 843 can have a disk 845 that is received within the inner tubular member 830 and moves (rotates) therein between a closed position (FIG. 36) in which the disk 845 closes off the inside center lumen of the inner tubular member 830. FIG. 37 shows the valve member 840 in the open position.

The HME bypass flow path is defined when the valve member 840 is in the closed position, which closes off the main flow path, and the breathing gas flowing through the HME unit 800 flows through the second opening 834 through the HME 200 and then flows back through the first opening 832 into the first main conduit 811 to the patient.

When the valve member 840 is in the open position, the breathing gas flows through the second part 820 axially through the inner tubular member 830 to the first part 810 and even though the HME bypass flow path remains fully open (since there are no valves in that direct flow path), the breathing gas flows axially along the main flow path due to the resistance of the HME 200 and the torturous path of the HME bypass flow path.

### HME Unit According to Eighth Embodiment

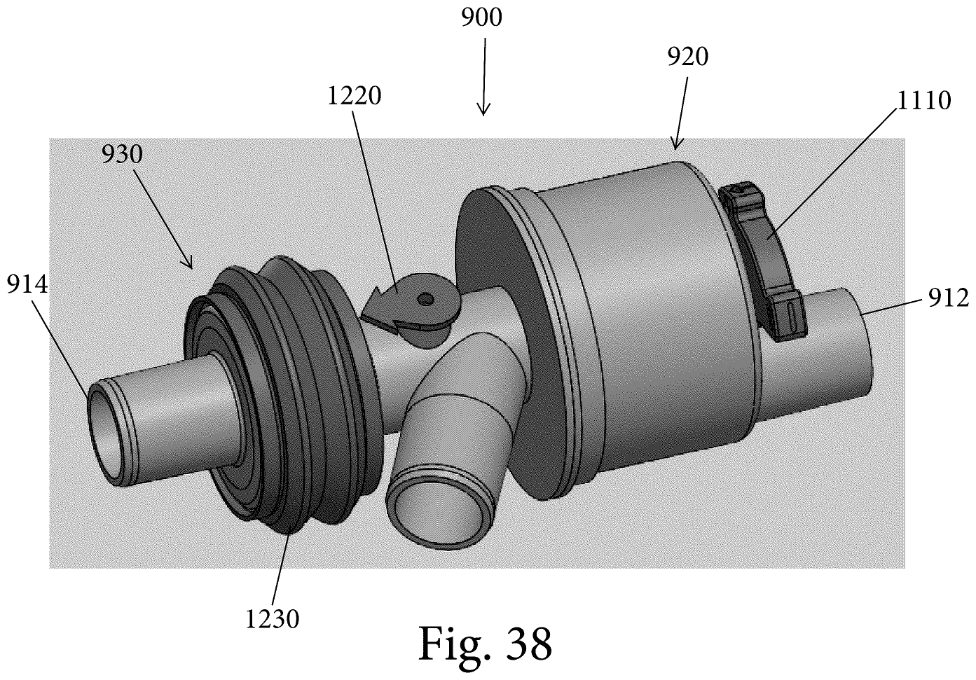

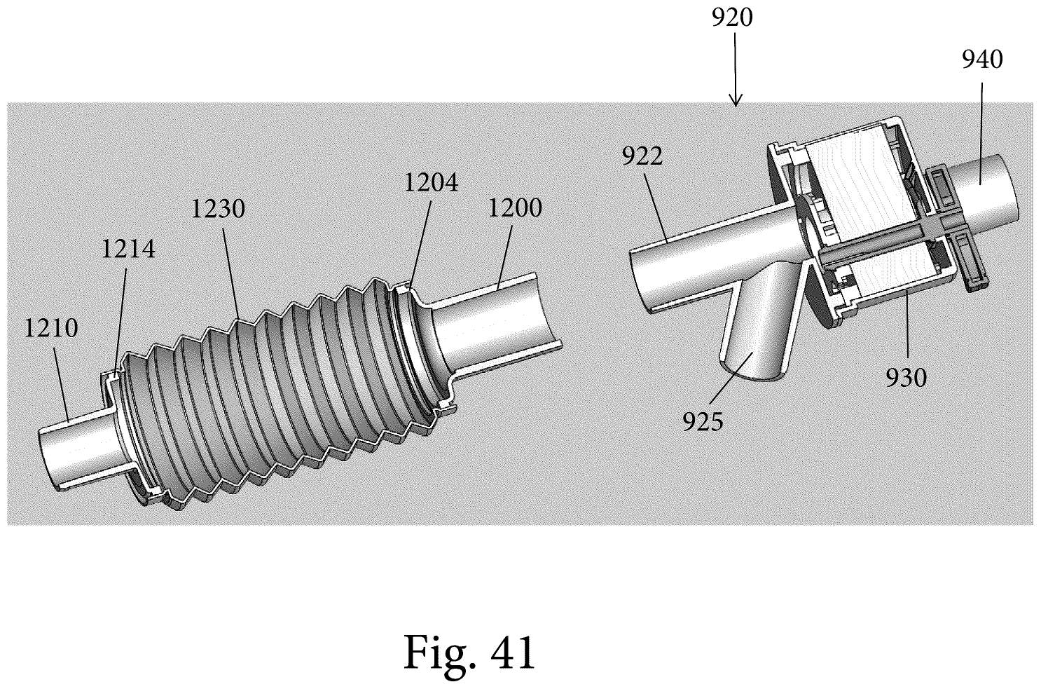

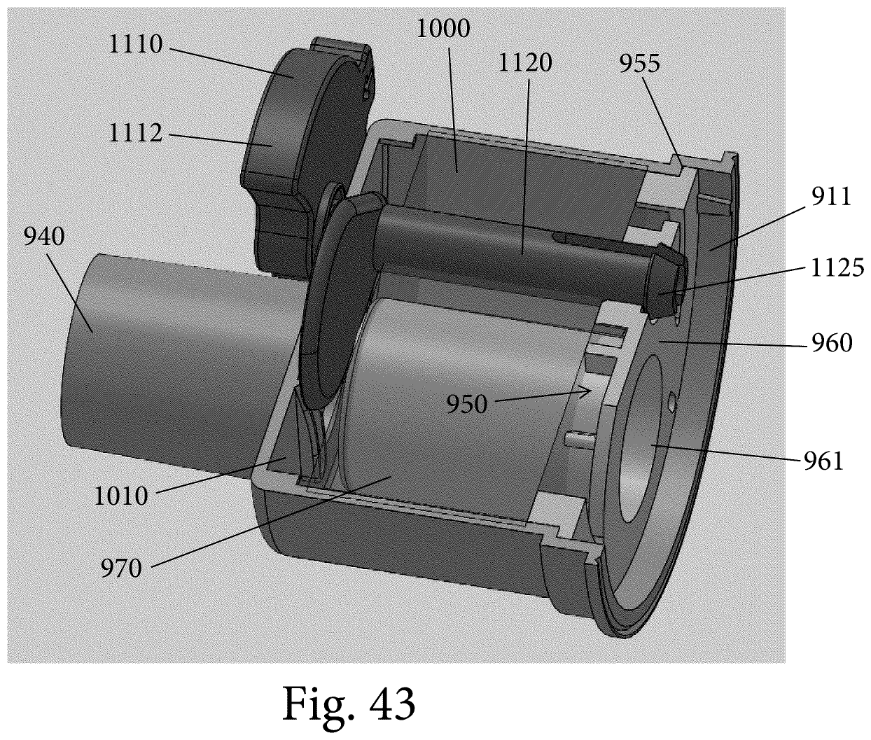

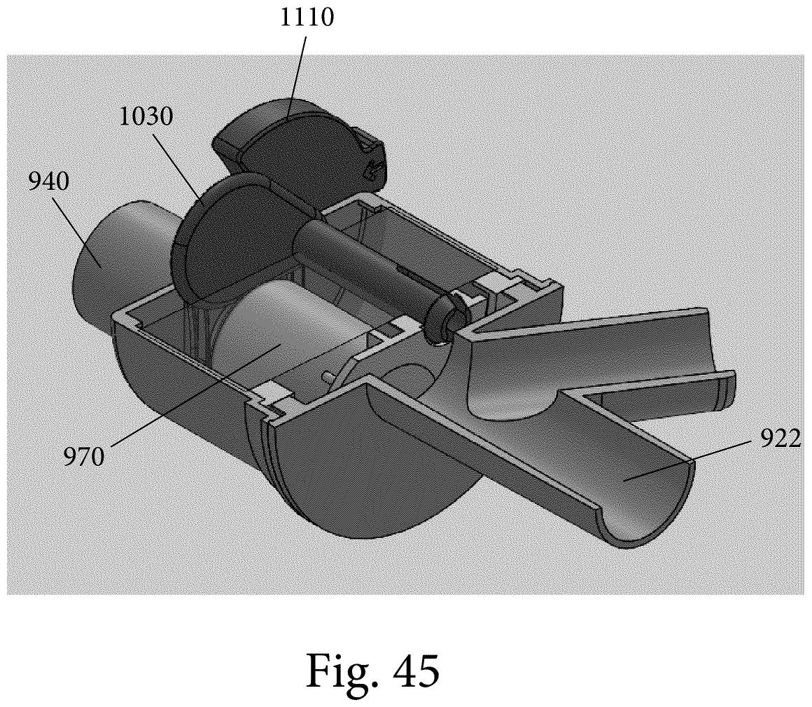

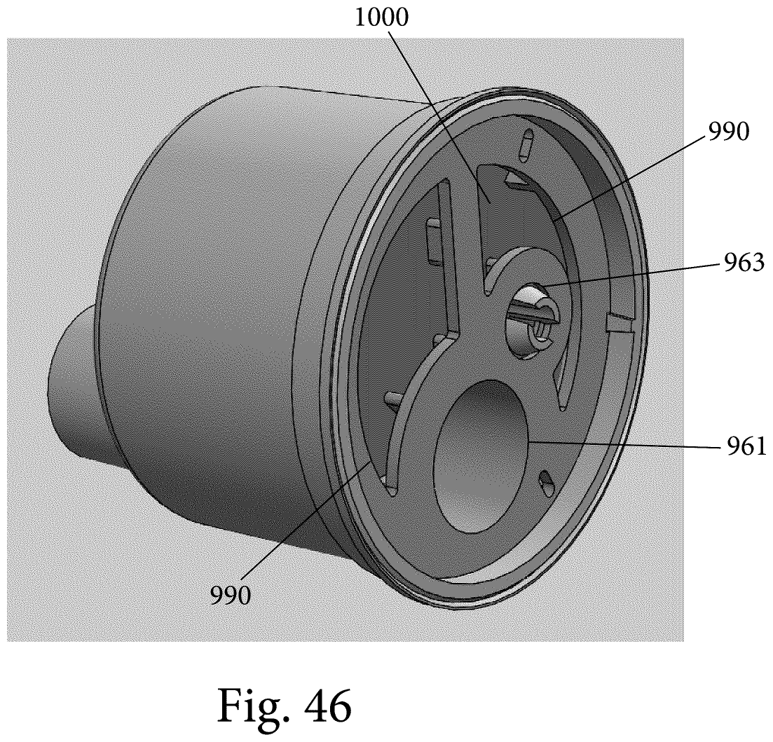

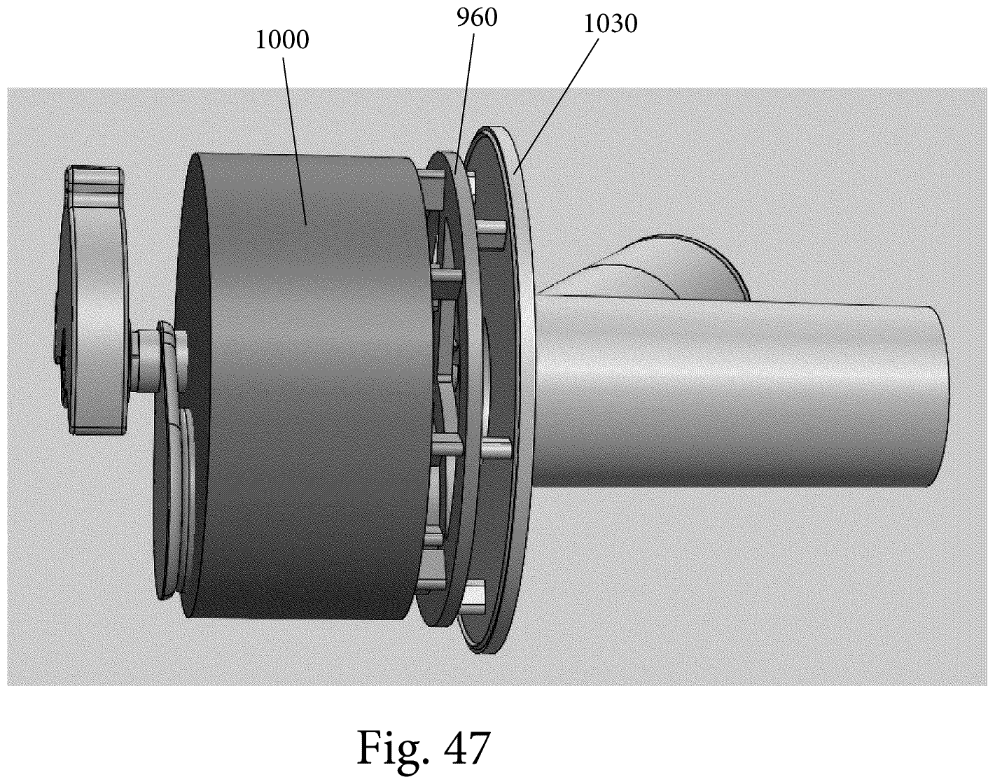

FIGS. 38-48 illustrate an HME unit 900 according to an eighth embodiment which is similar to the HME units 700, 800.

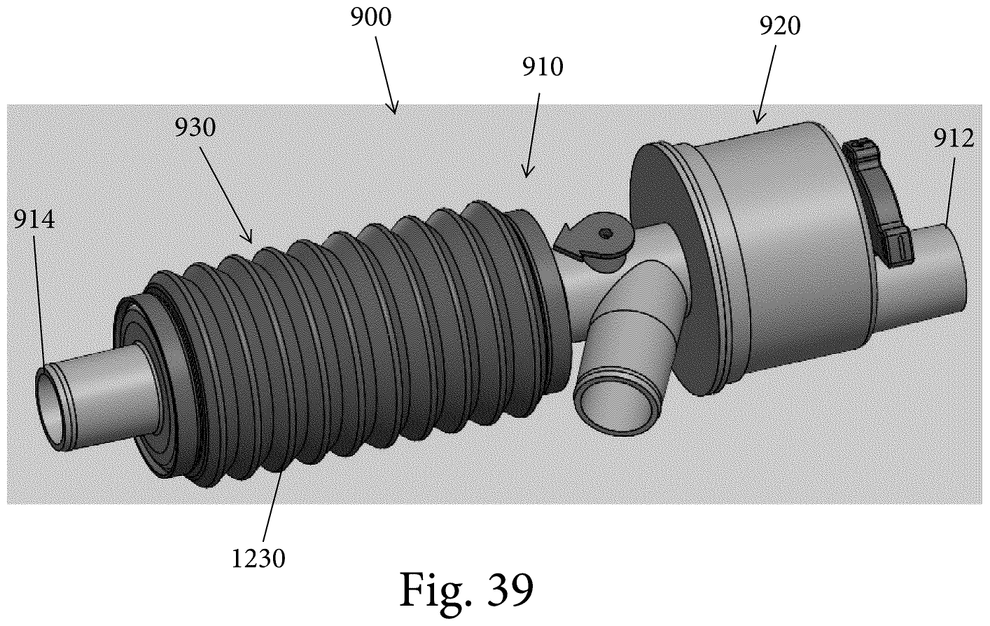

The HME unit 900 includes an overall main housing, generally indicated at 910, that includes a number of open ports. The housing 910 generally has a first end 912 and an opposing second end 914. The second end 914 can be attached to a source of fluid, while the first end 912 can be attached to a patient interface such as a breathing mask or breathing tube. In the illustrated embodiment, the fluid thus flows from left to right in FIGS. 38 and 39.

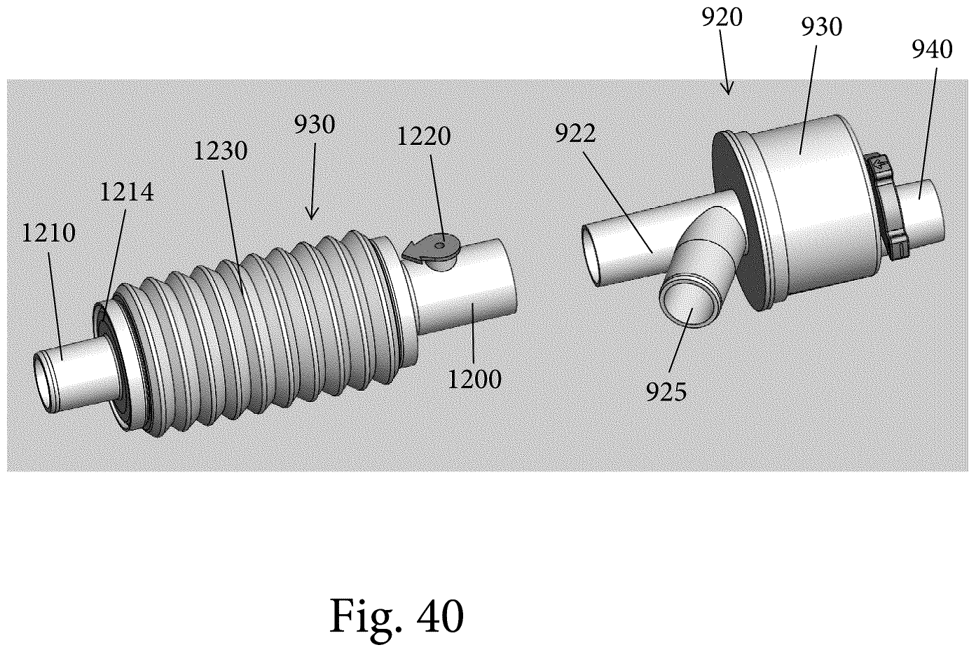

The housing 910 is formed of a number of individual parts that are assembled together as best shown in FIGS. 33 and 34. For example, the housing 910 can include a first part 920 and a second part 930 as shown in FIG. 40.

The first part 920 defines the first end 912 and can be a molded structure. The first part 920 includes at one end an inlet conduit (port) 922 that receives inhalation gas. The first part 920 also defines an HME bypass flow path as described herein. The first part 920 itself has a hollow housing 930 that can be cylindrical in shape.

The inlet conduit 922, in the illustrated embodiment, is part of a wye connector. The wye connector includes an exhalation side port 925 that is at an angle (e.g., less than 90 degrees) to the inlet conduit 922. The inlet conduit 922 thus receives gas for inhalation, while the exhalation side port 925 receives exhalation gas for exhalation. At the opposite end of the housing 930, a connector conduit (port) 940 is provided. The connector conduit 940 can be axially aligned with inlet conduit 922 or can be offset therefrom.

Within the hollow interior of the housing 930, an internal wall structure 950 is provided and is sealed to the inside of the housing 930 near one end thereof. As shown, the housing 930 can include an annular shoulder that defines an annular ledge 955. The internal wall structure 950 comprises a cylindrical plate 960 that has a shoulder that seats against and seals to the annular ledge 955. The cylindrical plate 960 can be ultrasonically sealed to the housing 930 or can be attached using other traditional techniques. The outer face of the plate 960 is thus spaced inward from the one end of the housing 930 so as to define a header space 911.

The internal wall structure 950 has an inner conduit 970 that protrudes outwardly from an inner face of the plate 960. The plate 960 has a first opening 961 that forms an entrance into the inner conduit 970. Gas flowing into the first opening 961 thus flows directly into the inner conduit 970 and then into the connector conduit 940 that can be connected to other equipment. The first opening 961 can have a circular shape.

The plate 960 further includes one or more HME openings 990. In the illustrated embodiment, there are two HME openings 990. Each HME opening 990 is arcuate shaped and the two HME openings 990 can have different sizes and shapes.

The plate 960 can also include a second opening 963 that can be a circular hole. The second opening 963 can have a smaller diameter as shown.

The hollow interior of the housing 910 functions as an HME compartment in which HME media 1000 is disposed. The HME media 1000 can be generally cylindrical in shape to be complementary to the cylindrical shape of the housing 910. As described herein, the HME media 1000 includes passageways or bores that receive and permit passage of internal component such as the inner conduit 970. The inner conduit 970 which is integral to the plate 960 can be inserted into and through the bore of the media 1000. As shown in the figures, the HME media 1000 does not fill up the entire hollow interior of the housing 910. Instead, a first end of the HME media 1000 seats against the inner face of the plate, while the other second end is offset from the other end of the housing 910 so as to define a valve compartment 1010. The inner conduit 970 extends slightly beyond the second end of the HME media 1000 and thus gas flowing through the inner conduit 970 flows into the valve compartment 1010 and can flow directly into the connector conduit 940.

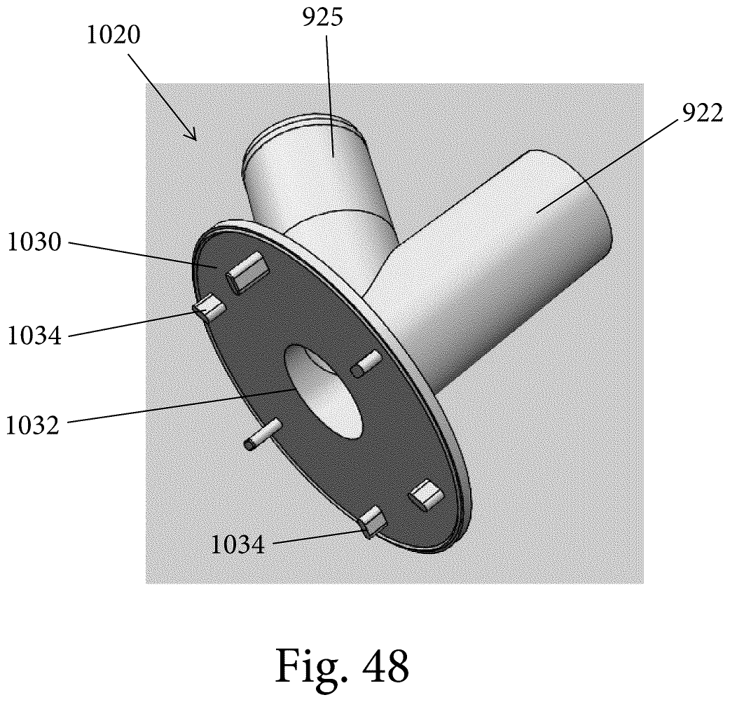

The first part 920 further includes a cap 1020 that is best shown in FIG. 48. The cap 1020 is sealingly attached to the first end of the housing 930. The cap 1020 includes a plate or disc 1030 that has a center hole 1032. When the cap 1020 is attached to the first end of the housing 910, the open header space 911 is formed between the inner face of the disc 1030 and the plate 960. The header space 911 can be an open cylindrical shaped space. Along the inner face of the disc 1030 there are a plurality of protrusions or posts 1034 that function as standoffs and contact the outer face of the plate 960 so as to ensure that the disc 1030 is spaced from the plate 960, thereby preserving the header space 911.

Similar to the plate 960, the cap 1020 can be attached to the housing 910 using any number of traditional techniques including but not limited to ultrasonic welding, etc.

The cap 1020 also includes one or more integral conduit and in particular, includes the inlet conduit 922 (wye connector). As mentioned, the wye connector includes the exhalation side port 925 that is at an angle to the inlet conduit 922. Both the inlet conduit 922 and the exhalation side port 925 are in fluid communication with the center hole 1032. When the cap 1020 is attached to the housing 930, the center hole 1032 is axially aligned with the inner conduit 970 and thus gas flowing through the center hole 1032 can flow axially into the inner conduit 970.

The first part 920 also includes a rotatable valve 1100 that is partially disposed in the valve compartment 1010. The rotatable valve 1100 includes a valve actuator 1110, a valve shaft 1120, and a valve member 1130. The valve actuator 1110 is located exterior to the housing 930 and is accessible and rotatable by the user to cause the valve 1100 to move between a fully open position and a fully closed position as described below.

The valve shaft 1120 passes through another bore formed through the HME media 1000 and is rotatable therein. As shown, the valve shaft 1120 is an elongated structure and can have a cylindrical shape. A distal end of the valve shaft 1120 has a shaft coupler 1125 that facilitates a rotatable coupling between the valve shaft 1120 and the plate 960. For example, a snap fit can result between the valve shaft 1120 and the plate 960 as by receiving the shaft coupler 1125 through the second opening 963; however, the valve shaft 1120 freely rotates relative to the plate 960. As shown, the valve shaft 1120 and the inner conduit 970 are parallel to one another but are spaced apart.

The compressible and pliable nature of the HME media 1000 permits insertion of the valve shaft 1120 and the shaft coupler 1125.

The valve member 1130 is integral to the valve shaft 1120 and extends radially outward therefrom and thus rotates with the valve shaft 1120. The valve member 1130 can comprise a paddle shaped valve structure that is formed perpendicular to a longitudinal axis of the valve shaft 1120. The valve member 1130 has a size and shape that allows it to completely cover the end of the inner conduit 970 located within the valve compartment 1010. The valve member 1130 is thus located within the valve compartment 1010 and moves therein between the fully closed position in which the valve member 1130 completely covers and occludes the inner conduit 970 and the fully open position in which the valve member 1130 is displaced from the end of the inner conduit 970 resulting in the inner conduit 970 being completely open.

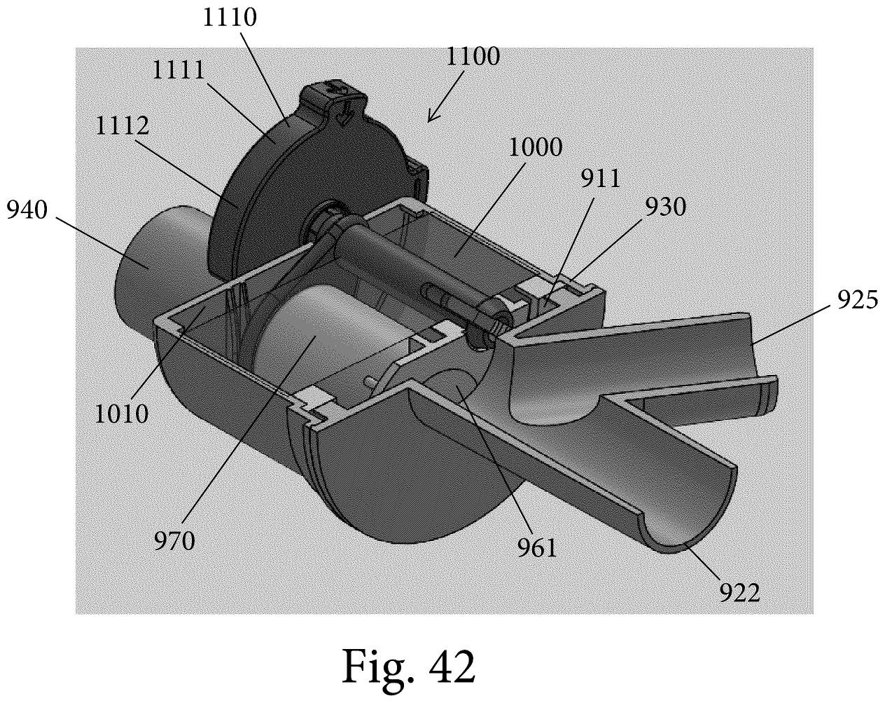

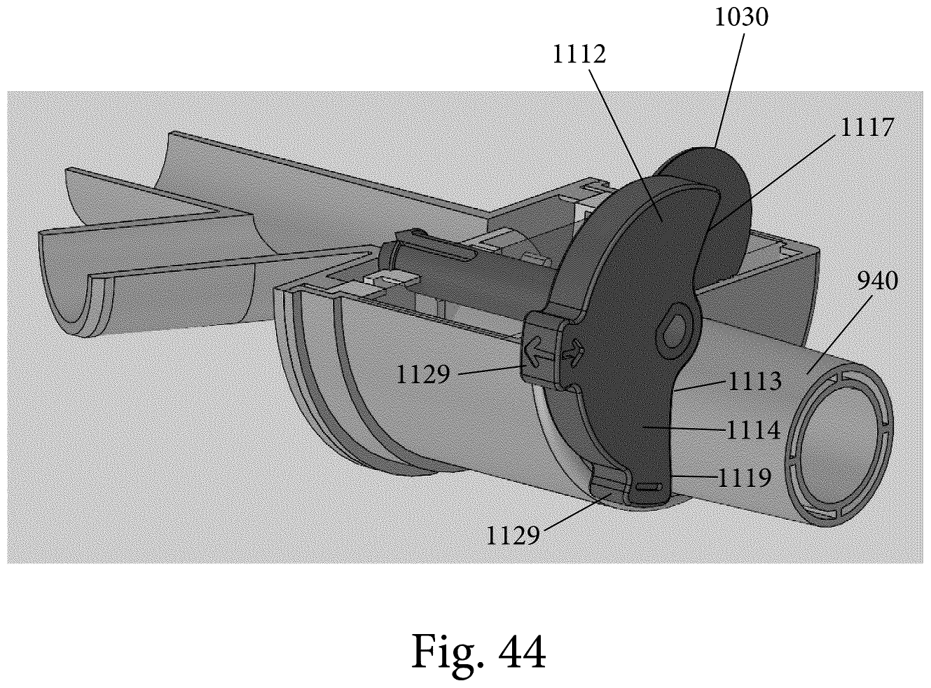

The valve actuator 1110 is located at a proximal end of the valve shaft 1120. The valve actuator 1110 is disposed proximate to the connector conduit 940 and selectively engages the connector conduit 940 in both the fully closed position and the fully open position. The valve actuator 1110 is thus located outside of the housing 930 (adjacent one end) and has a first wing portion 1112 and a second wing portion 1114. The valve actuator 1110 has an outer edge 1111 that faces away from the connector conduit 940 and an opposite inner edge 1113 that faces toward and selectively contacts the connector conduit 940. The inner edge 1113 can be thought of as including a first inner edge section 1117 that is located within the first wing portion 1112 and a second inner edge section 1119 that is located within the second wing portion 1114. Each of the first inner edge section 1117 and the second inner edge section 1119 can have a concave shape that complements the curvature of the connector conduit 940 which is tubular in nature. In other words, when the first inner edge section 1117 contacts the connector conduit 940, the valve actuator 1110 is in a position that represents the fully closed position of the valve (FIG. 42) and conversely, when the second inner edge section 1119 contacts the connector conduit 940, the valve actuator 1110 is in a position that represents the fully open position of the valve (FIG. 44). The arcuate contours of these two sections 1117, 1119 thus allows the valve actuator 1110 to seat flush against and circumferentially around the tube shaped connector conduit 940. This makes it easier for the user in that it eliminates the guess work as to where the fully closed position is for the valve actuator and where the fully open position is for the valve actuator. Instead, the user simply rotates the valve actuator 1110 in one direction until it stops due to contact with the connector conduit 940 and that represents one of the valve positions (fully open or fully closed) and conversely, the user rotates the valve actuator 1110 in the opposite direction until it stops due to contact with the connector conduit 940 and that represents the other of the valve positions (fully open or fully closed).

The outer edge 1111 includes several features that facilitate rotation of the valve actuator 1110. For example, as shown, the outer edge 1111 includes one or more raised tabs 1129 that can be manipulated by the user to cause rotation of the valve actuator 1110. There can also be indicia on the valve actuator 1110 to indicate which end corresponds to the fully closed valve position and which end corresponds to the fully open valve position.

It will be appreciated that as the valve actuator 1110 rotates, the valve member 1130 rotates within the valve compartment 1010.

As mentioned, when the valve member 1130 is in the fully open position, the inner conduit 970 is fully open and this allows the axial flow of gas into inner conduit 970 and then into the connector conduit 940 (this defines a main flow path). The same is also true when gas is exhaled, gas flow axially into the connector conduit 940 and through the inner conduit 970.

The HME bypass flow path is as follows. When the valve is in the fully closed position and the valve member 1130 completely closes off the inner conduit 970, gas flowing into the first part 920 will not flow axially into the inner conduit 970 since the other end of the inner conduit 970 is closed and this would require compression of the existing gas in the inner conduit 970 which is not favored. Instead, the gas coming into the first part 920 will instead flow radially outward and flows enters into the HME openings 990. Gas entering into the HME openings 990 flows directly into the HME media 1000 and exits the HME media into the valve compartment 1010 and then flows into the connector conduit 940 where it exits the first part 920. In addition, gas being exhaled travels through the HME media 1000 when the valve member is in the closed position.

The second part 930 is configured to mate with the first part 920 and in particular, a friction fit or snap-fit or other type of connection can be made to connect the two parts. The second part 930 has a first end connector 1200 at one end and there is a second end connector 1210 at the opposite end. The first end connector 1200 is intended to and is configured to be coupled to the inlet conduit 922 as by a friction fit or other type of fit and thus includes a first end connector conduit 1202 that mates with the inlet conduit 922 in a sealed manner. The first end connector 1200 can include a first end cap 1204 which can be integral to the first end connector conduit 1200. The first end cap 1204 can have a cylindrical shape.

An expandable/collapsible corrugated tubing 1230 is coupled at one end to the first end cap 1204. In the expanded state, the interior space (volume) within the tubing 1230 is increased, while in the collapsed state, the interior space (volume) within the tubing 1230 is reduced.

The second end connector 1210 is intended to and is configured to be coupled to a patient interface (e.g., a face mask) or the like as by a friction fit or other type of fit and thus includes a second end connector conduit 1212. The second end connector 1210 can include a second end cap 1214 which can be integral to the first end connector conduit 1200. The second end cap 1214 can have a cylindrical shape.

The tubing 1230 is easily expanded by pulling the two end connector conduits 1202, 1212 in opposite directions and the tubing 1230 is contracted by pushing the two end connector conduits 1202, 1212 toward one another.

The first end connector 1200 also includes an MDI access port 1220 that is in fluid communication with the main first end connector conduit 1200. The MDI access port 1220 comprises a nozzle that has a top opening in which an MDI is placed in fluid communication and the nozzle has an exit that is directed toward the tubing 1230. The arrow on the top of the MDI access port 1220 points in the direction of the tubing 1230 to indicate the direction of flow of the medication (aerosol) being delivered into the MDI access port 1220 by the MDI device (not shown). By directing the medication into the chamber 1230, the medication mixes with the air within this chamber 1230 and then is delivered into the first part 920 and ultimately to the patient by means of a patient interface that is attached to the first part 920.

As with the other embodiments, the HME bypass flow path is configured to force all of the fluid (breathing gas) through the HME 1000 when the user selects this mode of operation as by manipulating (turning) the valve actuator 1110.

When the other operating mode is selected in which the main flow path is open and represents that primary flow path, the breathing gas flows primarily through the main flow path since even if the HME bypass flow path remains open (which it always is), the flow resistance of the HME 1000 and the natural resistance of the torturous HME bypass flow path causes essentially all of the breathing gas to flow axially (linearly) within the main flow path (defined through conduits 922, 970, 940.

It will be appreciated that when the MDI device is used, the valve member 760 is open to allow the injected medication to flow through the main flow path to the patient. A plug or the like can be inserted into the MDI access port 1220 to close off when not in use.

It is to be understood that like numerals in the drawings represent like elements through the several figures, and that not all components and/or steps described and illustrated with reference to the figures are required for all embodiments or arrangements.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. An HME unit for use in a breathing circuit, the HME unit comprising:
a first conduit;
an HME chamber containing an HME member, the first conduit in fluid communication with the HME chamber;
a second conduit in fluid communication with the HME chamber;
an inner core for reception in the HME chamber, the inner core having at least one first opening formed therein and at least one second opening formed therein and spaced from the at least one first opening with an intermediate area being located between the at least one first opening and the at least one second opening;
the HME member being disposed about the intermediate area of the inner core; and
a movable valve that has a valve member disposed within an interior bore of the inner core and being movable between a fully open position and a fully closed position in which the valve member occludes the interior bore of the inner core and is configured to force incoming breathing gas to flow through the at least one first opening across the HME member before flowing back through the at least one second opening and subsequently into the second conduit.

2. The HME unit of claim 1, wherein the HME chamber has a cylindrical shape and one end of the inner core is coupled to an inner surface of a proximal end wall of the HME chamber.

3. The HME unit of claim 2, wherein the one end of the inner core has an outwardly extending flange that seats against the inner surface.

4. The HME unit of claim 1, wherein the at least one first opening comprises a pair of first arcuate slots spaced apart from one another and contained in a sample transverse plane that is perpendicular to a longitudinal axis of the inner core.

5. The HME unit of claim 1, wherein the at least one second opening comprises a pair of second arcuate slots spaced apart from one another and contained in a sample transverse plane that is perpendicular to a longitudinal axis of the inner core.

6. The HME unit of claim 1, wherein the HME member has an annular shape.

7. The HME unit of claim 1, wherein the second conduit is part of an end plate that includes an end plate flange that seals against an edge of a side wall of the HME chamber.

8. The HME unit of claim 7, wherein the edge of the side wall comprises a lip.

9. The HME unit of claim 1, wherein the movable valve extends through an opening formed through the inner core.

10. The HME unit of claim 1, wherein the movable valve includes a disk shaped valve member that is attached to a bottom end of a post with a top end of the post being attached to an actuator that is disposed along an exterior of the HME chamber.

11. The HME unit of claim 10, wherein in the fully closed position, the disk shaped valve seals against an inner surface of the inner core and extends in a plane that is perpendicular to a longitudinal axis of the inner core.

12. The HME unit of claim 10, wherein in the fully open position, the disk shaped valve extends in a plane the is parallel to a longitudinal axis of the inner core.

13. The HME unit of claim 12, wherein the plane is coaxial to the longitudinal axis.

14. The HME unit of claim 9, wherein the opening formed in the inner core is located between the at least one first opening and the at least one second opening.

15. The HME unit of claim 1, wherein the inner core has a cylindrical shape.

16. The HME unit of claim 1, wherein the first conduit, the inner core and the second conduit are coaxial to one another.

17. An HME unit with an HME bypass flowpath for use in a breathing circuit, the HME unit comprising:
a housing including an inlet for receiving breathing gas and an outlet for delivering the breathing gas to a patient;
an HME chamber containing an HME member, the first conduit in fluid communication with the HME chamber;
a main flow path defined in the HME unit in which the breathing gas bypasses the HME chamber and HME member;
an HME flow path defined in the HME unit in which the breathing gas flows into the HME chamber into contact with the HME member;
a valve member disposed within the main flow path outside of the HME flow path and positionable in at least a first position in which the main flow path is fully open and a second position in which the main flow path is closed and the breathing gas flows into the HME chamber and is conducted across the HME member before rejoining the main flow path;
wherein in both the first position and the second position of the valve member, the HME flow path remains open.
